(19) United States
(12) Patent Application Publication
(10) Pub. No.: US 2012/0157453 A1
(43) Pub. Date: Jun. 21, 2012

(54) RAF INHIBITOR COMPOUNDS AND METHODS OF USE THEREOF

(75) Inventors: Ignacio Aliagas, South San Francisco, CA (US); Stefan Gradl, South San Francisco, CA (US); Janet Gunzner, South San Francisco, CA (US); Simon Mathieu, South San Francisco, CA (US); Joachim Rudolph, South San Francisco, CA (US); Zhaoyang Wen, South San Francisco, CA (US); Steven Mark Wenglowsky, Boulder, CO (US)

(73) Assignees: GENENTECH, INC., South San Francisco, CA (US); ARRAY BIOPHARMA INC., Boulder, CO (US)

(21) Appl. No.: 13/393,111

(22) PCT Filed: Aug. 27, 2010
(86) PCT No.: PCT/US2010/046970

§ 371 Date: Feb. 28, 2012

Related U.S. Application Data
(60) Provisional application No. 61/238,108, filed on Aug. 28, 2009.

(51) Int. Cl. A61K 31/519; A61K 31/52; C07D 487/04; C07D 495/04; C07D 491/048; A61P 13/12; A61P 35/00; A61P 25/28; A61P 9/00; A61P 29/00; A61P 25/06; A61P 25/00; C07D 473/34; A61K 31/5377

(52) U.S. Cl. 514/234.2; 544/277; 514/263.4; 544/262; 514/262.1; 544/280; 514/265.1; 544/254; 514/261.1; 544/278; 514/260.1; 544/250; 514/267; 514/263.37; 544/118

(57) ABSTRACT

Compounds of Formula II are useful for inhibition of Raf kinases. Methods of using compounds of Formula II and stereoisomers, tautomers, prodrugs and pharmaceutically acceptable salts thereof, for in vitro, in situ, and in vivo diagnosis, prevention or treatment of such disorders in mammalian cells, or associated pathological conditions are disclosed.

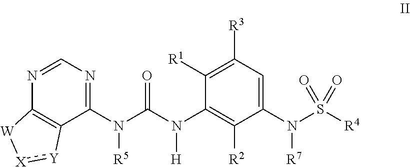

### PRIORITY OF INVENTION

This application claims priority under 35 U.S.C. 119(e) from U.S. Provisional Patent Application No. 61/238,108, filed 28 Aug. 2009, the content of which is incorporated herein in its entirety.

### BACKGROUND OF THE INVENTION

1. Field of the Invention

The present invention relates to novel compounds, to pharmaceutical compositions comprising the compounds, to a process for making the compounds and to the use of the compounds in therapy. More particularly, it relates to certain substituted compounds useful for inhibiting Raf kinase and for treating disorders mediated thereby.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example RAF-265, GSK-2118436, PLX-4032, PLX-3603, and XL-281. Other B-Raf inhibitors are also known, see for example, U.S. Patent Application Publication 2006/0189627, U.S. Patent Application Publication 2006/0281751, U.S. Patent Application Publication 2007/0049603, U.S. Patent Application Publication 2009/0176809, International Patent Application Publication WO 2007/002325, International Patent Application Publication WO 2007/002433, International Patent Application Publication WO 2008/028141, International Patent Application Publication WO 2008/079903, International Patent Application Publication WO 2008/079906 and International Patent Application Publication WO 2009/012283.

International Patent Application Publication WO 2006/066913, International Patent Application Publication WO 2008/028617 and International Patent Application Publication WO 2008/079909 also disclose kinase inhibitors.

### SUMMARY OF THE INVENTION

In one aspect, the invention relates to compounds that are inhibitors of Raf kinases, particularly B-Raf inhibitors. Certain hyperproliferative disorders are characterized by the overactivation of Raf kinase function, for example by mutations or overexpression of the protein. Accordingly, the compounds of the invention are useful in the treatment of hyperproliferative disorders, such as cancer.

More specifically, one aspect of the present invention provides compounds of

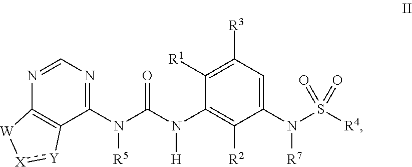

and stereoisomers, tautomers and pharmaceutically acceptable salts thereof, wherein R¹, R², R³, R⁴, R⁵, R⁷, W, X, and Y are as defined herein.

Another aspect of the present invention provides compounds of Formula I:

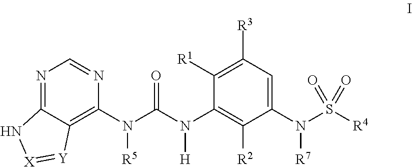

and stereoisomers, tautomers and pharmaceutically acceptable salts thereof, wherein R¹, R², R³, R⁴, R⁵, R⁷, X, and Y are as defined herein.

Another aspect of the present invention provides methods of preventing or treating a disease or disorder modulated by B-Raf, comprising administering to a mammal in need of such treatment an effective amount of a compound of this invention or a stereoisomer, tautomer prodrug or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating a disease or disorder modulated by B-Raf, comprising administering to a mammal in need of such treatment an effective amount of a compound of this invention or a stereoisomer, tautomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating cancer, comprising administering to a mammal in need of such treatment an effective amount of a compound of this invention, or a stereoisomer, tautomer, prodrug or pharmaceutically acceptable salt thereof, alone or in combination with one or more additional compounds having anti-cancer properties.

Another aspect of the present invention provides methods of preventing or treating cancer, comprising administering to a mammal in need of such treatment an effective amount of a compound of this invention, or a stereoisomer, tautomer or pharmaceutically acceptable salt thereof, alone or in combination with one or more additional compounds having anti-cancer properties.

Another aspect of the present invention provides a method of treating a hyperproliferative disease in a mammal comprising administering a therapeutically effective amount of a compound of this invention to the mammal.

Another aspect of the present invention provides methods of preventing or treating kidney disease, comprising administering to a mammal in need of such treatment an effective amount of a compound of this invention, or a stereoisomer, tautomer, prodrug or pharmaceutically acceptable salt thereof, alone or in combination with one or more additional compounds. Another aspect of the present invention provides methods of preventing or treating polycystic kidney disease, comprising administering to a mammal in need of such treatment an effective amount of a compound of this invention, or a stereoisomer, tautomer, prodrug or pharmaceutically acceptable salt thereof, alone or in combination with one or more additional compounds.

Another aspect of the present invention provides the compounds of the present invention for use in therapy.

Another aspect of the present invention provides the compounds of the present invention for use in the treatment of a hyperproliferative disease. In a further embodiment, the hyperproliferative disease may be cancer (or still further, a specific cancer as defined herein).

Another aspect of the present invention provides the compounds of the present invention for use in the treatment of a kidney disease. In a further embodiment, the kidney disease may be polycystic kidney disease.

Another aspect of the present invention provides the use of a compound of this invention in the manufacture of a medicament for the treatment of a hyperproliferative disease. In a further embodiment, the hyperproliferative disease may be cancer (or still further, a specific cancer as defined herein).

Another aspect of the present invention provides the use of a compound of this invention in the manufacture of a medicament for the treatment of a kidney disease. In a further embodiment, the kidney disease may be polycystic kidney disease.

Another aspect of the present invention provides the use of a compound of the present invention in the manufacture of a medicament, for use as a B-Raf inhibitor in the treatment of a patient undergoing cancer therapy.

Another aspect of the present invention provides the use of a compound of the present invention in the manufacture of a medicament, for use as a B-Raf inhibitor in the treatment of a patient undergoing polycystic kidney disease therapy.

Another aspect of the present invention provides a pharmaceutical composition comprising a compound of the present invention for use in the treatment of a hyperproliferative disease.

Another aspect of the present invention provides a pharmaceutical composition comprising a compound of the present invention for use in the treatment of cancer.

Another aspect of the present invention provides a pharmaceutical composition comprising a compound of the present invention for use in the treatment of polycystic kidney disease.

Another aspect of the present invention provides a pharmaceutical composition comprising a compound of this invention, a stereoisomer, tautomer, prodrug or a pharmaceutically acceptable salt thereof, and a pharmaceutically acceptable carrier or excipient.

Another aspect of the present invention provides a pharmaceutical composition comprising a compound of this invention or a pharmaceutically acceptable salt thereof, and a pharmaceutically acceptable carrier or excipient.

Another aspect of the present invention provides intermediates for preparing compounds of Formulas I-Ic and II. Certain compounds of Formulas I-Ic and II may be used as intermediates for other compounds of Formulas I-Ic and II.

Another aspect of the present invention includes methods of preparing, methods of separation, and methods of purification of the compounds of this invention.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” includes linear or branched-chain radicals of carbon atoms. In one example, the alkyl radical is one to six carbon atoms (C₁-C₆). In other examples, the alkyl radical is C₁-C₅, C₁-C₄ or C₁-C₃. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. Other examples of alkyl groups include 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂) and 3,3-dimethyl-2-butyl (—CH(CH₃)C(CH₃)₃. The abbreviations are sometimes used in conjunction with elemental abbreviations and chemical structures, for example, methanol (“MeOH”) or ethanol (“EtOH”).

Additional abbreviations used throughout the application include, for example, benzyl (“Bn”), phenyl (“Ph”) and acetyl (“Ac”).

The following terms are abbreviated: dimethylsulfoxide (“DMSO”), dimethylformamide (“DMF”), dichloromethane (“DCM”) and tetrahydrofuran (“THF”).

The term “alkenyl” refers to linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to six carbon atoms (C₂-C₆). In other examples, the alkenyl radical is C₂-C₅, C₂-C₄ or C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, hexa-1,3-dienyl.

The term “alkoxy” refers to a radical of the formula —O-alkyl.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to six carbon atoms (C₂-C₆). In other examples, the alkynyl radical is C₂-C₅, C₂-C₄ or C₂-C₃. Examples include, but are not limited to, ethynyl (—C≡CH), prop-1-ynyl (—C≡CCH₃), prop-2-ynyl (propargyl, CH₂C≡CH), but-1-ynyl, but-2-ynyl and but-3-ynyl.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 6 carbon atoms (C₃-C₆). In other examples, cycloalkyl is C₃-C₄ or C₃-C₅. In other examples, the cycloalkyl group, as a monocycle, is C₃-C₆ or C₅-C₆. In another example, the cycloalkyl group, as a bicycle, is C₇-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane, and bicyclo[3.2.2]nonane.

The terms “heterocyclic” or “heterocycle” or “heterocyclyl” refers to a saturated or a partially unsaturated (i.e., having one or more double and/or triple bonds within the ring) cyclic group in which at least one ring atom is a heteroatom independently selected from nitrogen, oxygen, and sulfur, the remaining ring atoms being carbon. In one embodiment, heterocyclyl includes saturated or partially unsaturated 4-6 membered heterocyclyl groups, another embodiment includes 5-6 membered heterocyclyl groups. The heterocyclyl group may be optionally substituted with one or more substituents described herein. Exemplary heterocyclyl groups include, but are not limited to, oxiranyl, aziridinyl, thiiranyl, azetidinyl, oxetanyl, thietanyl, 1,2-dithietanyl, 1,3-dithietanyl, pyrrolidinyl, piperidinyl, dihydropyridinyl, tetrahydropyridinyl, morpholinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, imidazolidinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Heterocycles include 4 to 6 membered rings containing one or two heteroatoms selected from oxygen, nitrogen and sulfur.

The term “heteroaryl” refers to an aromatic cyclic group in which at least one ring atom is a heteroatom independently selected from nitrogen, oxygen and sulfur, the remaining ring atoms being carbon. Heteroaryl groups may be optionally substituted with one or more substituents described herein. In one example, heteroaryl includes 5-6 membered heteroaryl groups. Other examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, and furopyridinyl. Heteroaryls includes 5 to 6 membered aromatic rings containing one, two or three heteroatoms selected from oxygen, nitrogen and sulfur.

“Halogen” refers to F, Cl, Br or I.

The abbreviation “TLC” stands for thin layer chromatography.

The terms “treat” or “treatment” refer to therapeutic, prophylactic, palliative or preventative measures. In one example, treatment includes therapeutic and palliative treatment. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound of the present invention that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term cancer may be used generically to include various types of cancer or specifically (as listed above).

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound of the invention.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

The term “mammal” means a warm-blooded animal that has or is at risk of developing a disease described herein and includes, but is not limited to, guinea pigs, dogs, cats, rats, mice, hamsters, and primates, including humans.

The terms “compound of this invention,” and “compounds of the present invention”, “compounds of Formula II,” unless otherwise indicated, include compounds of Formulas I, Ia, Ib, Ic and II, stereoisomers, tautomers, solvates, metabolites, salts (e.g., pharmaceutically acceptable salts) and prodrugs thereof. Unless otherwise stated, structures depicted herein are also meant to include compounds that differ only in the presence of one or more isotopically enriched atoms. For example, compounds of Formulas I, Ia, Ib, Ic, wherein one or more hydrogen atoms are replaced deuterium or tritium, or one or more carbon atoms are replaced by a ¹³C— or ¹⁴C-enriched carbon are within the scope of this invention.

B-Raf Inhibitor Compounds

The present invention provides compounds, and pharmaceutical formulations thereof, that are potentially useful in the treatment of diseases, conditions and/or disorders modulated by B-Raf.

One embodiment of this invention provides compounds of Formula II:

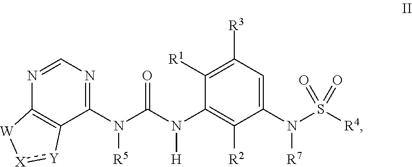

stereoisomers, tautomers, prodrugs and pharmaceutically acceptable salts thereof, wherein:

the dotted line represents an optional double bond;

W is NR⁶, O or S;

X is N or CR⁸;

Y is NR⁹ or CR⁹;

R¹ and R² are independently selected from hydrogen, halogen, CN, C₁-C₃ alkyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl or NR¹⁰R¹¹, wherein the cycloalkyl, alkyl, alkenyl, alkynyl, phenyl and heteroaryl are optionally substituted with OR¹², halogen, phenyl, C₃-C₄ cycloalkyl, or C₁-C₄ alkyl optionally substituted with halogen;

R⁵ is hydrogen, C₁-C₃ alkyl, C₂-C₃ alkenyl, C₂-C₃ alkynyl or C₃-C₅ cycloalkyl, wherein R⁵ is optionally substituted with halogen;

R⁶ is hydrogen or C₁-C₃ alkyl;

R⁷ is hydrogen, C₁-C₃ alkyl, C₂-C₃ alkenyl, C₂-C₃ alkynyl or C₃-C₅ cycloalkyl, wherein R⁷ is optionally substituted with halogen;

R⁸ is hydrogen, halogen, oxo, thioxo, C₁-C₆ alkoxy or C₁-C₆ alkyl, wherein each C₁-C₆ alkoxy and C₁-C₃ alkyl is optionally substituted with halogen, OR¹³, SR¹³, NR¹³R¹⁴, C₃-C₆ cycloalkyl, 4-6 membered heterocyclyl, 5-6 membered heteroaryl or phenyl;

R⁹ is hydrogen, halogen, C₁-C₆ alkoxy, C₁-C₆ alkyl, 4-6 membered heterocyclyl, 5-6 membered heteroaryl, phenyl or C₃-C₆ cycloalkyl, wherein said alkoxy, alkyl, heterocyclyl, heteroaryl, phenyl and cycloalkyl are independently optionally substituted with halogen, OR¹⁵, SR¹⁵, NR¹⁵R¹⁶, C₃-C₆ cycloalkyl, 4-6 membered heterocyclyl, 5-6 membered heteroaryl, phenyl or C₁-C₆ alkyl optionally substituted with halogen; or

R⁸ and R⁹ are taken together with the atoms to which they are attached to form a fused 5-6 membered heterocyclyl or C₅-C₆ cycloalkyl;

R¹⁰ and R¹¹ is independently hydrogen or C₁-C₆ alkyl, optionally substituted with halogen; or

R¹⁰ and R¹¹ are independently taken together with the atom to which they are attached to form a 3-6 membered heterocyclyl, optionally substituted with halogen, oxo or C₁-C₃ alkyl;

R¹² is hydrogen or C₁-C₆ alkyl optionally substituted with halogen;

R¹³ and R¹⁴ are independently hydrogen or C₁-C₆ alkyl optionally substituted with halogen; or

R¹³ and R¹⁴ are independently taken together with the atom to which they are attached to form a 3-6 membered heterocyclyl optionally substituted with halogen, oxo or C₁-C₃ alkyl; and

R¹⁵ and R¹⁶ are independently hydrogen or C₁-C₆ alkyl optionally substituted with halogen; or

R¹⁵ and R¹⁶ are independently taken together with the atom to which they are attached to form a 3-6 membered heterocyclyl optionally substituted with halogen, oxo or C₁-C₃ alkyl.

Another embodiment includes compounds of Formula I:

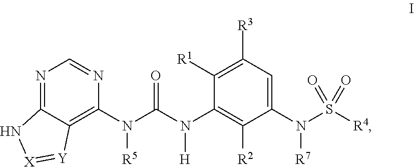

stereoisomers, tautomers, prodrugs and pharmaceutically acceptable salts thereof, wherein:

X is N or CR⁸;

Y is N or CR⁹;

R¹ and R² are independently selected from hydrogen, halogen, CN, C₁-C₃ alkyl and C₁-C₃ alkoxy;

R³ is hydrogen, halogen or C₁-C₃ alkyl;

R⁴ is C₃-C₅ cycloalkyl, C₁-C₆ alkyl, C₂-C₆ alkenyl, C₂-C₆ alkynyl, phenyl, a 5-6 membered heteroaryl or NR¹⁰R¹¹, wherein the cycloalkyl, alkyl, alkenyl, alkynyl, phenyl and heteroaryl are optionally substituted with OR¹², halogen, phenyl, C₃-C₄ cycloalkyl, or C₁-C₄ alkyl optionally substituted with halogen;

R⁵ is hydrogen, C₁-C₃ alkyl, C₂-C₃ alkenyl, C₂-C₃ alkynyl or C₃-C₅ cycloalkyl, wherein R⁵ is optionally substituted with halogen;

R⁷ is hydrogen, C₁-C₃ alkyl, C₂-C₃ alkenyl, C₂-C₃ alkynyl or C₃-C₅ cycloalkyl, wherein R⁷ is optionally substituted with halogen;

R⁸ is hydrogen, C₁-C₆ alkoxy or C₁-C₃ alkyl, wherein each C₁-C₆ alkoxy and C₁-C₃ alkyl is optionally substituted with halogen, OR¹³, SR¹³, NR¹³R¹⁴, C₃-C₆ cycloalkyl, 4-6 membered heterocyclyl, 5-6 membered heteroaryl or phenyl;

R⁹ is hydrogen, C₁-C₆ alkoxy or C₁-C₃ alkyl, wherein each C₁-C₆ alkoxy and C₁-C₃ alkyl is optionally substituted with halogen, OR¹⁵, SR¹⁵, NR¹⁵R¹⁶, C₃-C₆ cycloalkyl,

R¹⁰ and R¹¹ is independently hydrogen or C₁-C₆ alkyl, optionally substituted with halogen; or

R¹⁰ and R¹¹ are independently taken together with the atom to which they are attached to form a 3-6 membered heterocyclyl, optionally substituted with halogen, oxo or C₁-C₃ alkyl;

R¹² is hydrogen or C₁-C₆ alkyl optionally substituted with halogen;

R¹³ and R¹⁴ are independently hydrogen or C₁-C₆ alkyl optionally substituted with halogen; or

R¹³ and R¹⁴ are independently taken together with the atom to which they are attached to form a 3-6 membered heterocyclyl optionally substituted with halogen, oxo or C₁-C₃ alkyl; and

R¹⁵ and R¹⁶ are independently hydrogen or C₁-C₆ alkyl optionally substituted with halogen; or

R¹⁵ and R¹⁶ are independently taken together with the atom to which they are attached to form a 3-6 membered heterocyclyl optionally substituted with halogen, oxo or C₁-C₃ alkyl.

In certain embodiments, W is NR⁶. In certain embodiments, W is NH.

In certain embodiments, W is O.

In certain embodiments, W is S.

In certain embodiments, W is NR⁶, X is N and Y is CR⁹. In certain embodiments, W is NH, X is N and Y is CR⁹.

In certain embodiments, W is NR⁶, X is CR⁸ and Y is NR⁹. In certain embodiments, W is NH, X is C═O or C═S, and Y is NH.

In certain embodiments, W is O, X is CR⁸ and Y is CR⁹.

In certain embodiments, W is S, X is CR⁸ and Y is CR⁹.

In certain embodiments, W is NR⁶, and X and Y are N. In certain embodiments, W is NH, and X and Y are N.

In certain embodiments, X is N. In certain embodiments, X is N and Y is CR⁹.

In certain embodiments, X and Y are N.

In certain embodiments, X is CR⁸. In certain embodiments, X is CR⁸ and Y is N.

In certain embodiments, X is CR⁸ and Y is CR⁹.

In certain embodiments, R¹, R² and R³ are independently selected from hydrogen, halogen or C₁-C₃ alkyl; R⁴ is C₃-C₄ cycloalkyl or C₁-C₆ alkyl optionally substituted with OH, halogen or C₃-C₄ cycloalkyl; R⁵ is hydrogen or C₁-C₃ alkyl; R⁷ is hydrogen or C₁-C₃ alkyl; R⁸ is hydrogen; and R⁹ is hydrogen, C₁-C₆ alkoxy or C₁-C₃ alkyl, wherein each C₁-C₆ alkoxy and C₁-C₃ alkyl is optionally substituted with OH.

In certain embodiments, R¹ and R² are independently selected from hydrogen, halogen, CN, C₁-C₃ alkyl or C₁-C₃ alkoxy.

In certain embodiments, R¹, R² and R³ are independently selected from hydrogen, halogen or C₁-C₃ alkyl.

In certain embodiments, R¹, R² and R³ are independently selected from hydrogen, F, Cl or methyl.

In certain embodiments, R¹ and R³ are independently selected from hydrogen, halogen or C₁-C₃ alkyl, and R² is Cl. In certain embodiments, R¹ and R³ are independently selected from hydrogen, F, Cl and methyl, and R² is Cl.

In certain embodiments, R¹ is hydrogen, halogen, CN, C₁-C₃ alkyl or C₁-C₃ alkoxy.

In certain embodiments, R¹ is hydrogen.

In certain embodiments, R¹ is halogen. In certain embodiments, R¹ is F or Cl.

In certain embodiments, R¹ is C₁-C₃ alkyl. In certain embodiments, R¹ is methyl.

In certain embodiments, R² is hydrogen, halogen, CN, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl.

In certain embodiments, R² is Cl.

In certain embodiments, R² is hydrogen.

In certain embodiments, R² is C₁-C₃ alkoxy. In certain embodiments, R² is methoxy.

In certain embodiments, R³ is hydrogen, halogen or C₁-C₃ alkyl.

In certain embodiments, R³ is hydrogen.

In certain embodiments, R³ is halogen. In certain embodiments, R³ is F or Cl.

In certain embodiments, R¹ and R² are F and R³ is hydrogen.

In certain embodiments, R¹ is F and R² is Cl and R³ is hydrogen.

In certain embodiments, R¹ is Cl and R² is F and R³ is hydrogen.

In certain embodiments, R¹ is F and R² and R³ are hydrogen.

In certain embodiments, R¹ and R³ are hydrogen and R² is F.

In certain embodiments, R² and R³ are F and R¹ is hydrogen.

In certain embodiments, R¹ is Cl and R² and R³ are hydrogen.

In certain embodiments, R¹, R² and R³ are F.

In certain embodiments, R¹ is F and R² is methyl and R³ is hydrogen.

In certain embodiments, R¹ is methyl and R² is F and R³ is hydrogen.

In certain embodiments, R¹ is F and R² and R³ are hydrogen.

In certain embodiments, R¹ is Cl and R² and R³ are hydrogen.

In certain embodiments, R² is F and R¹ and R³ are hydrogen.

In certain embodiments, R² is methoxy, R¹ is F and R³ is hydrogen.

In certain embodiments, the residue:

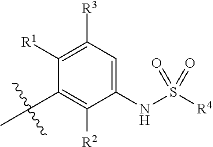

of Formulas I-II, wherein the wavy line represents the point of attachment of the residue in Formulas I-II, is selected from:

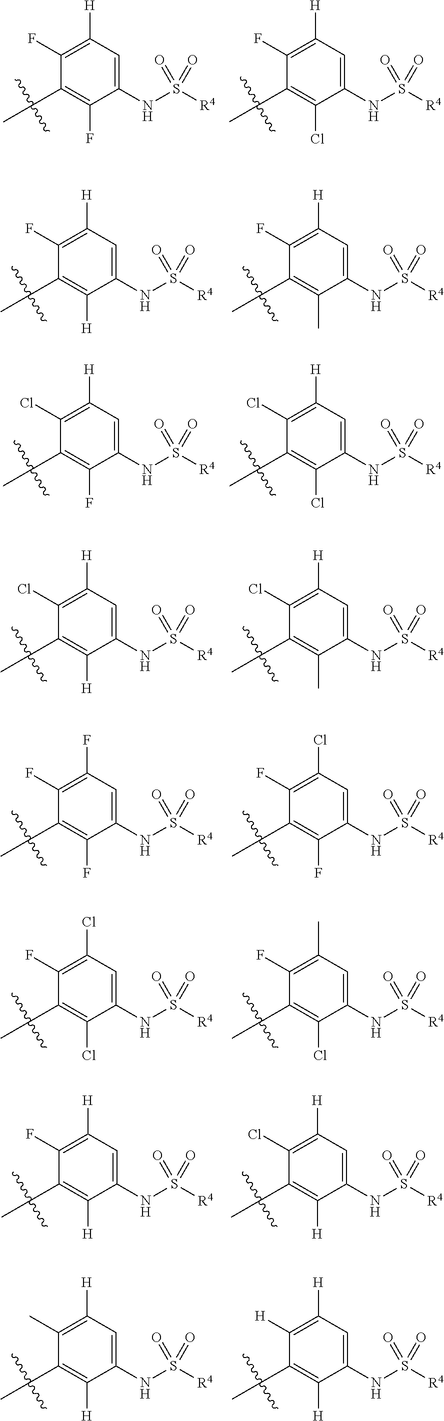

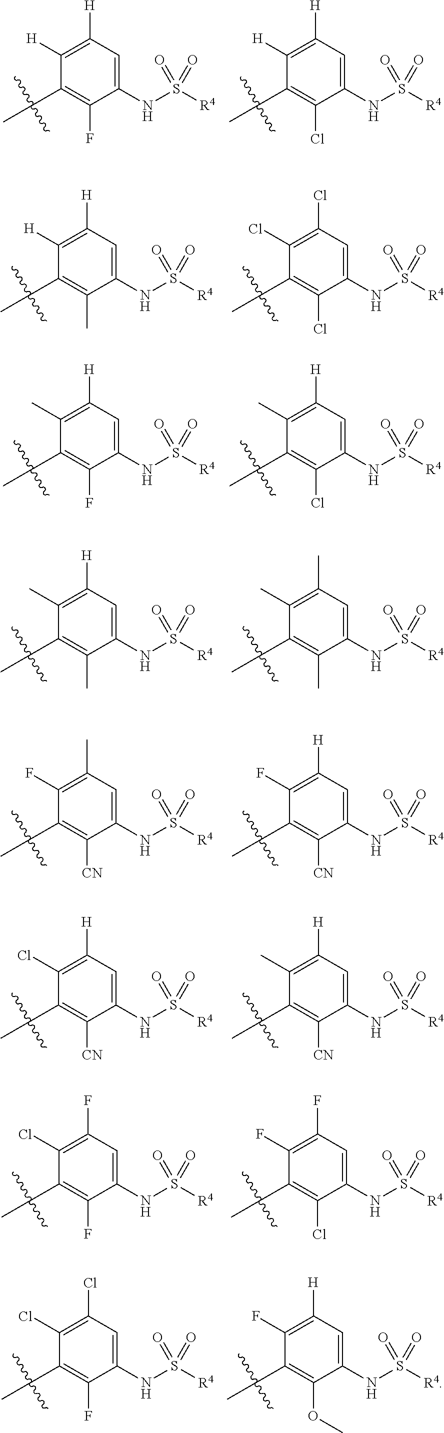

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl or NR¹⁰R¹¹, wherein the cycloalkyl, alkyl, alkenyl, alkynyl, phenyl and heteroaryl are optionally substituted with OR¹², halogen, phenyl, C₃-C₄ cycloalkyl, or C₁-C₄ alkyl optionally substituted with halogen.

In certain embodiments, R⁴ is C₃-C₄ cycloalkyl, C₁-C₆ alkyl optionally substituted with halogen or C₃-C₄ cycloalkyl, or NR¹⁰R¹¹. In certain embodiments, R¹⁰ and R¹¹ are independently selected from hydrogen and C₁-C₅ alkyl.

In certain embodiments, R⁴ is C₃-C₅ cycloalkyl, C₁-C₆ alkyl, C₂-C₆ alkenyl or C₂-C₆ alkynyl, wherein the cycloalkyl, alkyl, alkenyl and alkynyl are optionally substituted with OR⁸, halogen or C₃-C₄ cycloalkyl.

In certain embodiments, R⁴ is cyclopropyl, ethyl, propyl, butyl, isobutyl, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —N(CH₃)₂, or pyrrolidine.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl, —NHCH₂CH₂CH₃, —N(CH₃)CH₂CH₃, —N(CH₃)₂, or pyrrolidine.

In certain embodiments, R⁴ is cyclopropyl, propyl, butyl, isobutyl, —CH₂Cl, —CH₂CF₃, —CH₂CH₂CH₂F, —CH₂CH₂CF₃, cyclopropylmethyl or —NHCH₂CH₂CH₃.

In certain embodiments, R⁴ is propyl, butyl, isobutyl, —CH₂CH₂CH₂F, —CH₂CH₂CF₃ or cyclopropylmethyl.

In certain embodiments, R⁴ is C₃-C₅ cycloalkyl or C₁-C₆ alkyl optionally substituted with OH, halogen or C₃-C₄ cycloalkyl.

In certain embodiments, R⁴ is C₃-C₅ cycloalkyl. In certain embodiments, R⁴ is C₃-C₄ cycloalkyl. In certain embodiments, R⁴ is cyclopropyl or cyclobutyl.

In certain embodiments, R⁴ is C₁-C₆ alkyl. In certain embodiments, R⁴ is ethyl, propyl, butyl or isobutyl. In certain embodiments, R⁴ is propyl.

In certain embodiments, R⁴ is C₁-C₆ alkyl optionally substituted with halogen. In certain embodiments, R⁴ is —CF₃, —CH₂Cl, —CH₂CF₃, —CH₂CH₂CH₂F, —CH₂CH₂CF₃, —CF₂CF₃ or —CF₂CF₂CF₃.

In certain embodiments, R⁴ is C₁-C₆ alkyl optionally substituted with OH, halogen or C₃-C₄ cycloalkyl. In certain embodiments, R⁴ is cyclopropylmethyl (—CH₂-cyclopropyl) or cyclobutylmethyl (—CH₂-cyclobutyl). In certain embodiments, R⁴ is cyclopropylmethyl (—CH₂-cyclopropyl).

In certain embodiments, R⁴ is C₁-C₆ alkyl optionally substituted with phenyl. In certain embodiments, R⁴ is phenylmethyl.

In certain embodiments, R⁴ is phenyl optionally substituted with OR¹², halogen, C₃-C₄ cycloalkyl, or C₁-C₄ alkyl optionally substituted with halogen. In certain embodiments, R⁴ is phenyl optionally substituted with halogen. In certain embodiments, R⁴ is phenyl optionally substituted with C₁-C₄ alkyl optionally substituted with halogen. In certain embodiments, R⁴ is phenyl optionally substituted with halogen and C₁-C₄ alkyl optionally substituted with halogen. In certain embodiments, R⁴ is phenyl. In certain embodiments, R⁴ is phenyl, 2-fluorophenyl, 3-fluorophenyl, 4-fluorophenyl, 2,5-difluorophenyl or 4-chloro-3-trifluoromethylphenyl.

In certain embodiments, R⁴ is a 5-6 membered heteroaryl optionally substituted with OR⁸, halogen, C₃-C₄ cycloalkyl or C₁-C₄ alkyl optionally substituted with halogen. In certain embodiments, R⁴ is a 5-6 membered heteroaryl optionally substituted with C₁-C₄ alkyl. In certain embodiments, R⁴ is a 5-6 membered heteroaryl, wherein the heteroaryl contains one or two heteroatoms selected from the group consisting of oxygen, nitrogen and sulfur. In certain embodiments, R⁴ is a 5-6 membered heteroaryl, wherein the heteroaryl is imidazolyl, furanyl, pyridinyl or thiophenyl. In certain embodiments, R⁴ is 1-methyl-1H-imidazol-4-yl, furan-2-yl, pyridin-2-yl, pyridin-3-yl or thiophen-2-yl.

In certain embodiments, R⁴ is NR¹⁰R¹¹. In certain embodiments, R¹⁰ and R¹¹ are independently selected from hydrogen and C₁-C₆ alkyl. In certain embodiments, R⁸ is hydrogen. In certain embodiments, R¹⁰ is C₁-C₆ alkyl. In certain embodiments, R¹⁰ is methyl, ethyl or propyl. In certain embodiments, R¹¹ is hydrogen or methyl. In certain embodiments, R⁴ is selected from the group consisting of —NHCH₂CH₃, —NHCH₂CH₂CH₃, —N(CH₃)CH₂CH₃ and —N(CH₃)₂.

In certain embodiments, R¹⁰ and R¹¹ together with the nitrogen to which they are attached form a 4 to 6 membered heterocyclic ring. In certain embodiments, R¹⁰ and R¹¹ together with the nitrogen to which they are attached form a 4 to 6 membered heterocyclic ring, wherein the heterocyclic ring contains one nitrogen heteroatom. In certain embodiments, R⁴ is pyrrolidine.

In certain embodiments, R⁴ is selected from propyl, cyclopropylmethyl, —CH₂CH₂CH₂F and phenyl. In a further embodiment, R⁴ is selected from propyl, cyclopropylmethyl and —CH₂CH₂CH₂F.

In certain embodiments, R⁵ is hydrogen or methyl.

In certain embodiments, R⁵ is hydrogen.

In certain embodiments, R⁷ is hydrogen or methyl.

In certain embodiments, R⁷ is hydrogen.

In certain embodiments, R⁸ is hydrogen, halogen, C₁-C₆ alkyl, C₁-C₆ alkoxy or thioxo, wherein said alkyl and alkoxy are independently optionally substituted with halogen.

In certain embodiments, R⁸ is hydrogen, methyl, thioxo, —CF₃ or bromo.

In certain embodiments, R⁸ is hydrogen or C₁-C₆ alkoxy.

In certain embodiments, R⁸ is hydrogen.

In certain embodiments, R⁹ is hydrogen, halogen, C₁-C₆ alkyl, C₁-C₆ alkoxy or 5-6 membered heterocyclyl, wherein said alkyl, alkoxy and heterocyclyl are independently optionally substituted by halogen, C₁-C₃ alkyl optionally substituted with halogen or OR¹⁵.

In certain embodiments, R⁹ is hydrogen, methoxy, ethoxy, —O(CH₂)₃OH, methyl, morpholinyl or pyrrolidinyl.

In certain embodiments, R⁸ and R⁹ are taken together with the atoms to which they are attached to form a fused C₅-C₆ cycloalkyl.

In certain embodiments, R⁸ and R⁹ are taken together with the atoms to which they are attached to form a fused cyclopentyl.

In certain embodiments, R⁹ is hydrogen or C₁-C₆ alkoxy optionally substituted by OH. In certain embodiments, R⁹ is hydrogen, ethoxy, methoxy, 3-hydroxypropoxy, or 2-hydroxyethoxy.

In certain embodiments of Formula I, R¹ and R² are F, R³ is hydrogen, R⁴ is propyl and R⁷ is hydrogen, such that the compounds have the structure of Formula Ia:

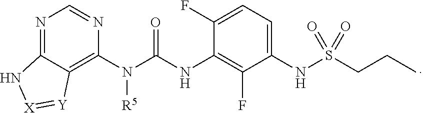

### Ia

In certain embodiments of Formula I, R¹ is Cl and R² is F, R³ is hydrogen, R⁴ is propyl and R⁷ is hydrogen, such that the compounds have the structure of Formula Ib:

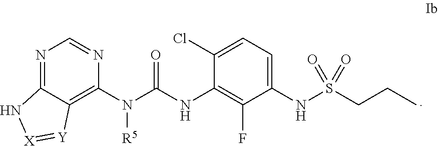

In certain embodiments of Formula I, R¹ is F and R² is Cl, R³ is hydrogen, R⁴ is propyl and R⁷ is hydrogen, such that the compounds have the structure of Formula Ic:

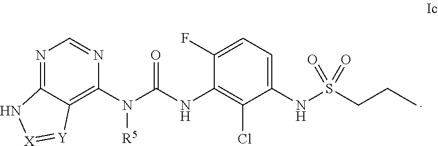

It will be appreciated that certain compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

It will also be appreciated that compounds of Formulas I-Ic and II include tautomeric forms. Tautomers are compounds that are interconvertible by tautomerization. This commonly occurs due to the migration of a hydrogen atom or proton, accompanied by the switch of a single bond and adjacent double bond. Tautomers of Formulas I-Ic and II may form at positions, including, but not limited to, the sulfonamide or R⁵ position depending on the substitution. The compounds of Formulas I-Ic and II are intended to include all tautomeric forms.

It will also be appreciated that certain compounds of Formulas I-Ic and II may be used as intermediates for further compounds of Formulas I-Ic and II.

It will be further appreciated that the compounds of the present invention may exist in unsolvated, as well as solvated forms with pharmaceutically acceptable solvents, such as water, ethanol, and the like, and it is intended that the invention embrace both solvated and unsolvated forms.

The term “prodrug” as used in this application refers to a precursor or derivative form of a compound of the invention that is less active or inactive compared to the parent compound or drug and is capable of being metabolized in vivo into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, N-methyl prodrugs (including N-methyl sulfonamide prodrugs), phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug.

Prodrugs of compounds of Formulas I-Ic and II may not be as active as the compounds of Formulas I-Ic and II in the assay as described in Example A. However, the prodrugs are capable of being converted in vivo into more active metabolites of compounds of Formulas I-Ic and II.

Synthesis of Compounds

Compounds of the present invention may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

For illustrative purposes, Schemes 1-8 show general methods for preparing the compounds of the present invention, as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

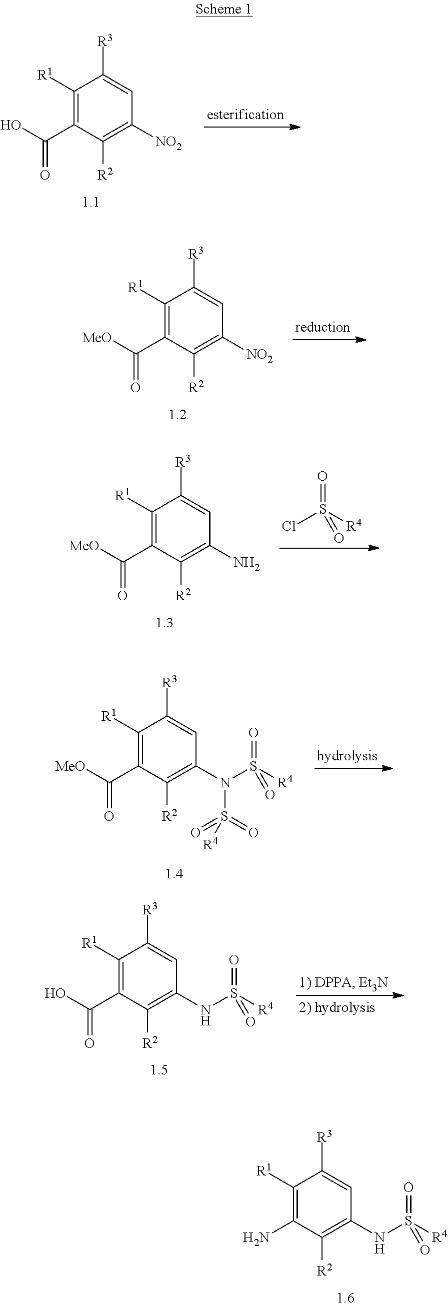

Scheme 1 shows a general method for preparing a compound 1.6, wherein R¹, R², R³ and R⁴ are as defined herein. A benzoic acid 1.1 is esterified to a methyl benzoate 1.2 by treatment with trimethylsilyl diazomethane in MeOH, or via Fischer esterification conditions, such as treatment with trimethylsilyl chloride (“TMSCl”) in MeOH. Reduction of nitro intermediate 1.2 to its amino analog 1.3 is performed using a standard condition, such as treatment with Pd/C and H₂. Bis-sulfonamide 1.4 is obtained by treatment of the aniline 1.3 with a sulfonyl chloride R⁴SO₂Cl in the presence of a base, such as NEt₃, in an organic solvent, such as dichloromethane (“DCM”). Hydrolysis of compound 1.4 is accomplished under basic conditions, such as aqueous NaOH, in the appropriate solvent system such as THF and/or MeOH, to provide a carboxylic acid 1.5. This compound in a suitable solvent, such as THF, is treated with diphenylphosphonic azide (“DPPA”) and a base, such as triethylamine (Curtius rearrangement conditions), and subsequently hydrolyzed to form an amine 1.6.

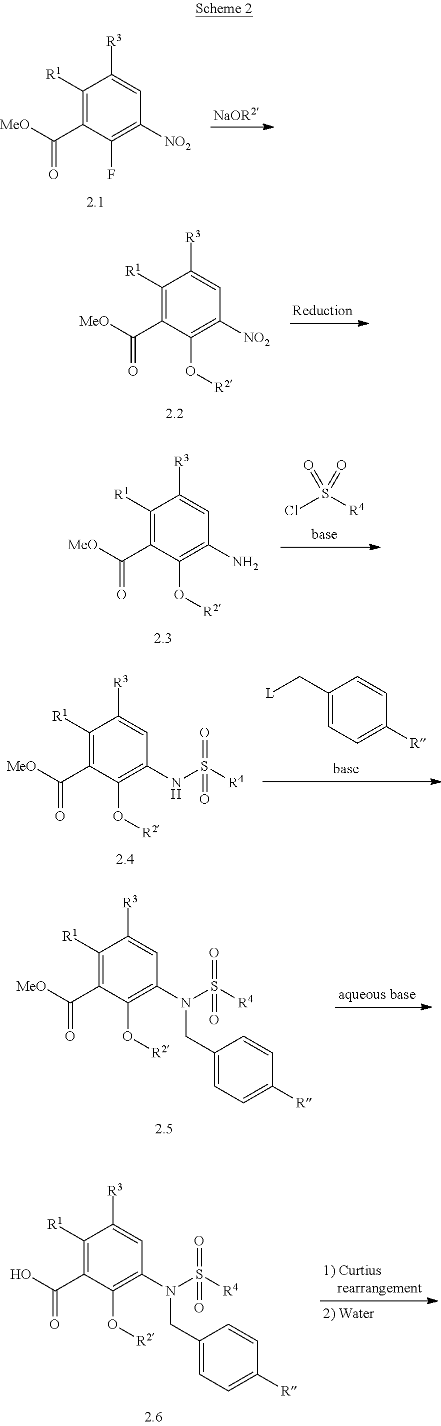

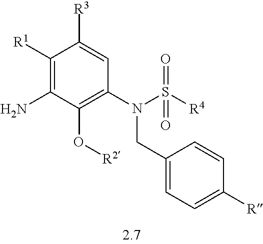

Scheme 2 describes the synthesis of aniline intermediates 2.7, wherein R¹, R^{2′}, R³ and R⁴ and R″ are as defined herein. A benzoic acid ester 2.1 is treated with an alkoxide NaOR²′ (wherein R²′ is C1-C3 alkyl) in an appropriate solvent, such as methanol, to form the ether intermediate 2.2. Reduction of the nitro group affords an aniline 2.3, which is reacted with a sulfonyl chloride R⁴SO₂Cl in the presence of base, such as pyridine, to give a sulfonamide intermediate 2.4. Benzylation with an optionally substituted benzyl halide, for example p-methoxybenzyl chloride, (wherein L is a leaving group such as chloro, bromo, iodo, triflate, tosylate; and R″ is hydrogen, C₁-C₃ alkyl or C₁-C₆ alkoxy; and in one example, R″ is hydrogen, in another example, R″ is OMe) in the presence of a base, such as sodium hydride, yields the protected sulfonamide ester 2.5, which is hydrolyzed with aqueous base, such as NaOH, to form the acid 2.6. In the last step, application of Curtius rearrangement conditions and subsequent hydrolysis gives the amino intermediate 2.7.

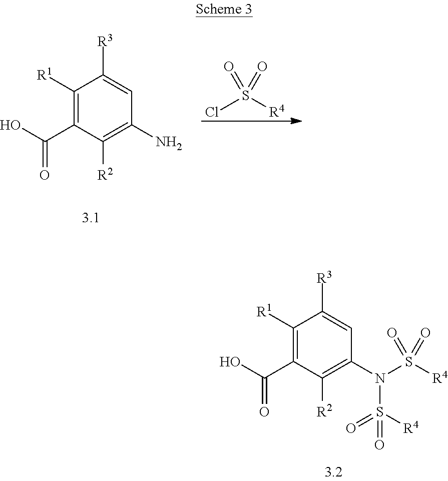

Scheme 3 shows a general method for preparing compounds 3.2, wherein R¹, R², R³ and R⁴ are as defined herein. An aniline 3.1 is sulfonylated in an organic solvent, such as DCM, in the presence of a base, such as NEt₃, to provide a compound 3.2.

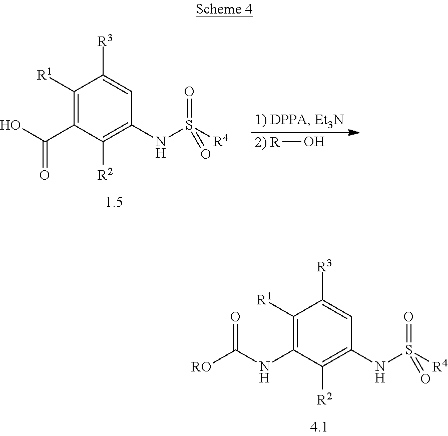

Scheme 4 shows a general method for preparing compounds 4.1, wherein R¹, R², R³ and R⁴ are as defined herein. A carboxylic acid 1.5 in a suitable solvent, such as THF, is treated with DPPA and a base, such as triethylamine, and subsequently treated with an alcohol (wherein R is C₁-C₆ alkyl or phenyl), such as phenol, to form a carbamate 4.1.

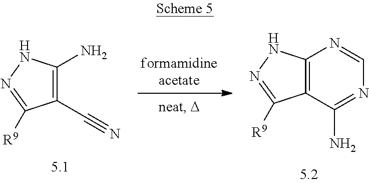

Scheme 5 illustrates a general method for preparing compounds 5.2. A carbonitrile derivative of Formula 5.1, for example prepared using a general method described in Scheme 5a, is thermally cyclized with formamidine acetate to form heterocyclic intermediate 5.2.

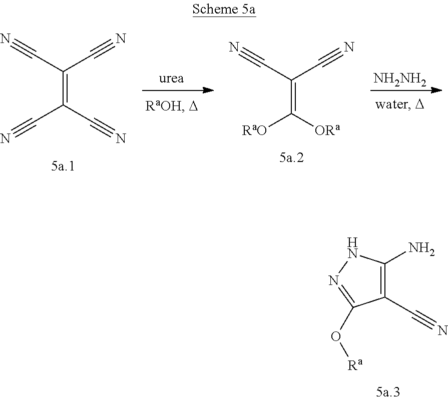

Scheme 5a illustrates a general method for preparing compounds of Formula 5a.3, a subset of Formula 5.1 compounds (wherein R⁹ is OR^{a} and R^{a} is C₁-C₆ alkyl optionally substituted by halogen, OR¹⁵, SR¹⁵, NR¹⁵R¹⁶, C₃-C₆ cycloalkyl, 4-6 membered heterocyclyl, 5-6 membered heteroaryl or phenyl). Tetracyanoethylene 5a.1 at elevated temperature, is reacted with urea and alcohol R^{a}OH, serving both as reactant and solvent, to form a dicyanoketene dialkyl acetal 5a.2. Subsequent reaction with hydrazine and water yields pyrazole intermediate 5a.3.

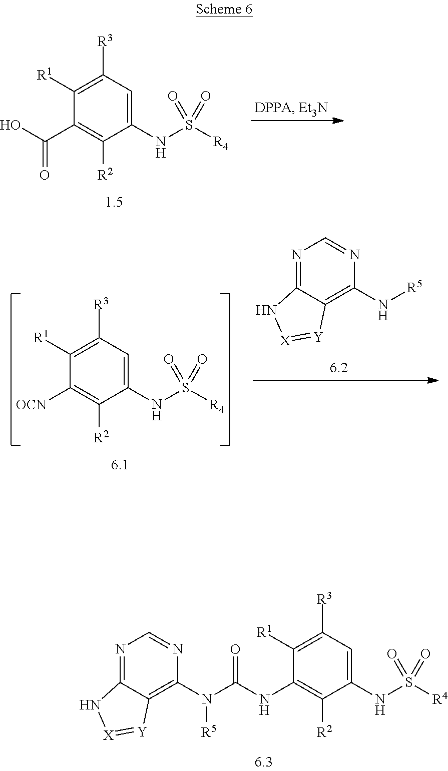

Scheme 6 shows a general method for preparing compounds 6.3, wherein R¹, R², R³, R⁴, R⁵, X and Y are as defined herein. A carboxylic acid 1.5 in an appropriate solvent, such as THF, is treated with DPPA and a base, such as triethylamine, to form an isocyanato intermediate 6.1. This intermediate is not isolated but further reacted, in the same pot, with a heterocyclic amine 6.2 to form compounds 6.3.

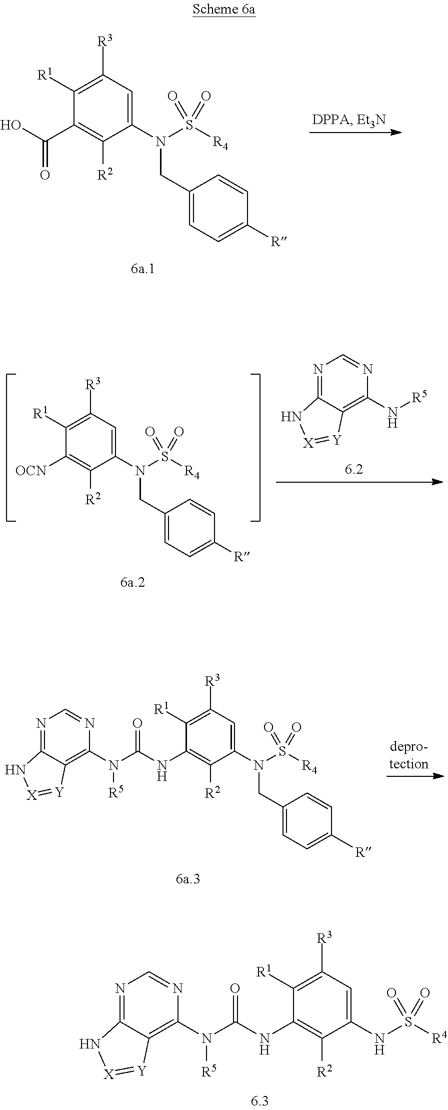

Scheme 6a shows a modification of Scheme 6 for preparing compounds 6.3. A protected carboxylic acid 6a.1 in an appropriate solvent, such as THF, is treated with DPPA and a base, such as triethylamine, to form an isocyanato intermediate 6a.2. The protecting group is either benzyl (R″═H) or methoxybenzyl (R″═OMe). The resulting intermediate is not isolated but further reacted, in the same pot, with a heterocyclic amine 6.2 to form compounds 6a.3. Compound 6a.3 is deprotected, for example using strong acid, such as trifluoroacteic acid, to form compounds 6.3.

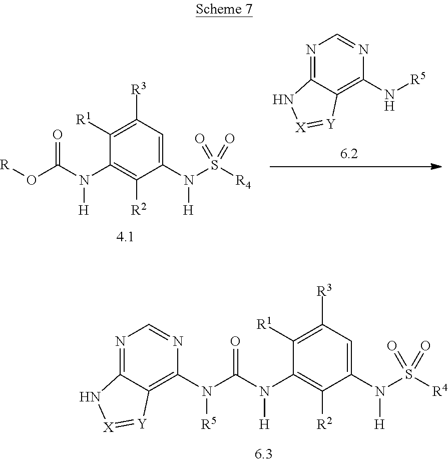

Scheme 7 illustrates another general method for preparing compounds 6.3, wherein R¹, R², R³, R⁴, R⁵, X and Y are as defined herein. A carbamate 4.1 is reacted in an appropriate solvent, such as DMSO, with a heterocyclic amine 6.2 to form compounds 6.3.

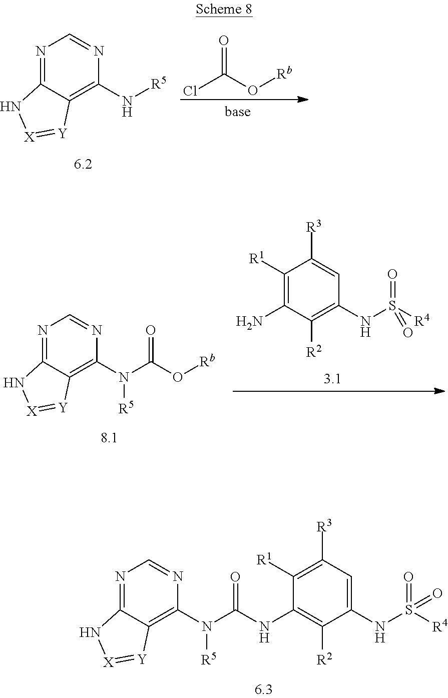

Scheme 8 illustrates another general method for preparing compounds 6.3, wherein R¹, R², R³, R⁴, R⁵, X and Y are as defined herein. A heterocyclic amine 6.2 in an appropriate solvent, such as THF, is treated with a base, such as cesium carbonate, and a carbamoyl chloride (wherein R^{b} is C₁-C₃ alkyl, phenyl or benzyl), for example phenyl- or benzyl-carbamoylchloride to form a carbamate 8.1. Further reaction with an aniline 3.1 furnishes compounds 6.3.

In preparing compounds of Formulas I-Ic and II, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-³³P]ATP into FSBA-modified wild-type MEK (see Example A).

Administration and Pharmaceutical Formulations

The compounds of the invention may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formulas I-Ic and II, or a stereoisomer or pharmaceutically acceptable salt thereof. In a further embodiment, the present invention provides a pharmaceutical composition comprising a compound of Formulas I-Ic and II, or a stereoisomer or pharmaceutically acceptable salt thereof, together with a pharmaceutically acceptable carrier or excipient.

Another embodiment of the present invention provides a pharmaceutical composition comprising a compound of Formulas I-Ic and II for use in the treatment of a hyperproliferative disease.

Another embodiment of the present invention provides a pharmaceutical composition comprising a compound of Formulas I-Ic and II for use in the treatment of cancer.

Another embodiment of the present invention provides a pharmaceutical composition comprising a compound of Formulas I-Ic and II for use in the treatment of kidney disease. A further embodiment of the present invention provides a pharmaceutical composition comprising a compound of Formulas I-Ic and II for use in the treatment of polycystic kidney disease.

Methods of Treatment with Compounds of the Invention

The invention includes methods of treating or preventing disease or condition by administering one or more compounds of this invention, or a stereoisomer or pharmaceutically acceptable salt thereof. In one embodiment, a human patient is treated with a compound of Formulas I-Ic and II, or a stereoisomer, tautomer or pharmaceutically acceptable salt thereof, and a pharmaceutically acceptable carrier, adjuvant, or vehicle in an amount to detectably inhibit B-Raf activity.

In another embodiment, a human patient is treated with a compound of Formulas I-Ic and II, or a stereoisomer, tautomer, prodrug or pharmaceutically acceptable salt thereof, and a pharmaceutically acceptable carrier, adjuvant, or vehicle in an amount to detectably inhibit B-Raf activity.

In another embodiment of the present invention, a method of treating a hyperproliferative disease in a mammal comprising administering a therapeutically effective amount of the compound of Formulas I-Ic and II, or a stereoisomer, tautomer, prodrug or pharmaceutically acceptable salt thereof, to the mammal is provided.

In another embodiment of the present invention, a method of treating a hyperproliferative disease in a mammal comprising administering a therapeutically effective amount of the compound of Formulas I-Ic and II, or a stereoisomer, tautomer or pharmaceutically acceptable salt thereof, to the mammal is provided.

In another embodiment of the present invention, a method of treating kidney disease in a mammal comprising administering a therapeutically effective amount of the compound of Formulas I-Ic and II, or a stereoisomer, tautomer, prodrug or pharmaceutically acceptable salt thereof, to the mammal is provided. In another embodiment of the present invention, a method of treating kidney disease in a mammal comprising administering a therapeutically effective amount of the compound of Formulas I-Ic and II, or a stereoisomer, tautomer or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formulas I-Ic and II, or a stereoisomer, tautomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment of the present invention provides the use of a compound of Formulas I-Ic and II, or a stereoisomer, tautomer or pharmaceutically acceptable salt thereof, in the manufacture of a medicament for the treatment of cancer.

In another embodiment, a method of treating or preventing cancer in a mammal in need of such treatment, wherein the method comprises administering to said mammal a therapeutically effective amount of a compound of Formulas I-Ic and II, or a stereoisomer, tautomer, prodrug or pharmaceutically acceptable salt thereof.

Another embodiment of the present invention provides the use of a compound of Formulas I-Ic and II, or a stereoisomer, tautomer, prodrug or pharmaceutically acceptable salt thereof, in the manufacture of a medicament for the treatment of cancer.

Another embodiment of the present invention provides the use of a compound of Formulas I-Ic and II, or a stereoisomer, tautomer, prodrug or pharmaceutically acceptable salt thereof, in the manufacture of a medicament for the treatment of kidney disease. Another embodiment of the present invention provides the use of a compound of Formulas I-Ic and II, or a stereoisomer, tautomer or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formulas I-Ic and II, or a stereoisomer, tautomer, prodrug or pharmaceutically acceptable salt thereof, alone or in combination with one or more additional compounds having anti-cancer properties.

In another embodiment, a method of preventing or treating cancer, comprising administering to a mammal in need of such treatment an effective amount of a compound of Formulas I-Ic and II, or a stereoisomer, tautomer or pharmaceutically acceptable salt thereof, alone or in combination with one or more additional compounds having anti-cancer properties.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

In another embodiment, a method of treating or preventing a disease or disorder modulated by B-Raf, comprising administering to a mammal in need of such treatment an effective amount of a compound of Formulas I-Ic and II, or a stereoisomer, tautomer or pharmaceutically acceptable salt thereof. Examples of such diseases and disorders include, but are not limited to, cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In another embodiment, a method of treating or preventing a disease or disorder modulated by B-Raf, comprising administering to a mammal in need of such treatment an effective amount of a compound of Formulas I-Ic and II, or a stereoisomer, tautomer, prodrug or pharmaceutically acceptable salt thereof.

In another embodiment of the present invention, a method of preventing or treating kidney disease, comprising administering to a mammal in need of such treatment an effective amount of a compound of Formulas I-Ic and II, or a stereoisomer, tautomer prodrug or pharmaceutically acceptable salt thereof, alone or in combination with one or more additional compounds. In another embodiment of the present invention, a method of preventing or treating polycystic kidney disease, comprising administering to a mammal in need of such treatment an effective amount of a compound of Formulas I-Ic and II, or a stereoisomer, tautomer prodrug or pharmaceutically acceptable salt thereof, alone or in combination with one or more additional compounds.

Another embodiment of the present invention provides the use of a compound of Formulas I-Ic and II, or a stereoisomer, tautomer or pharmaceutically acceptable salt thereof, in the manufacture of a medicament for the treatment of cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. In a further embodiment, the use of a compound of Formulas I-Ic and II in the manufacture of a medicament, for use as a b-Raf inhibitor in the treatment of a patient undergoing cancer therapy.

Another embodiment of the present invention provides the use of a compound of Formulas I-Ic and II, or a stereoisomer, tautomer, prodrug or pharmaceutically acceptable salt thereof, in the manufacture of a medicament for the treatment of cancer.

Another embodiment of the present invention provides the use of a compound of Formulas I-Ic and II, or a stereoisomer, tautomer, prodrug or pharmaceutically acceptable salt thereof, in the manufacture of a medicament for the treatment of polycystic kidney disease. In a further embodiment, the kidney disease is polycystic kidney disease.

Another embodiment of the present invention provides the compounds of Formulas I-Ic and II for use in therapy.

Another embodiment of the present invention provides the compounds of Formulas I-Ic and II for use in the treatment of a hyperproliferative disease. In a further embodiment, the hyperproliferative disease is cancer (as further defined and may be individually selected from those above).

Another embodiment of the present invention provides the compounds of Formulas I-Ic and II for use in the treatment of kidney disease. In a further embodiment, the kidney disease is polycystic kidney disease.

Combination Therapy

The compounds of this invention, stereoisomers, tautomers and pharmaceutically acceptable salts thereof may be employed alone or in combination with other therapeutic agents for treatment. The compounds of the present invention can be used in combination with one or more additional drugs, for example an anti-hyperproliferative, anti-cancer, or chemotherapeutic agent. The second compound of the pharmaceutical combination formulation or dosing regimen preferably has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®, Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhône-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) PI3k/AKT/mTOR pathway inhibitors, including GDC-0941 (2-(1H-Indazol-4-yl)-6-(4-methanesulfonyl-piperazin-1-ylmethyl)-4-morpholin-4-yl-thieno[3,2-d]pyrimidine), XL-147, GSK690693 and temsirolimus; (xi) Ras/Raf/MEK/ERK pathway inhibitors; and (xii) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

In order to illustrate the invention, the following Examples are included. However, it is to be understood that these Examples do not limit the invention and are only meant to suggest a method of practicing the invention. Persons skilled in the art will recognize that the chemical reactions described may be readily adapted to prepare a number of other compounds of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography purification was done on a Biotage system (Manufacturer: Dyax Corporation) having a silica gel column or on a silica SepPak cartridge (Waters) or on a Teledyne Isco Combiflash purification system using prepacked silica gel cartridges. ¹H-NMR spectra were recorded on a Bruker AVIII 400 MHz or Bruker AVIII 500 MHz or on a Varian 400 MHz NMR spectrometer.

¹H-NMR spectra were obtained as CDCl₃, CD₂Cl₂, CD₃OD, D₂O, DMSO-d₆, acetone-d₆ or CD₃CN solutions (reported in ppm), using tetramethylsilane (0.00 ppm) or residual solvent (CDCl₃: 7.25 ppm; CD₃OD: 3.31 ppm; D₂O: 4.79 ppm; DMSO-d₆: 2.50 ppm; d₆-acetone: 2.05 ppm; CD₃CN: 1.94 ppm) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

### Example A

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-³³P]ATP into FSBA-modified wild-type MEK. The 30-μL assay mixtures contained 25 mM Na Pipes, pH 7.2, 100 mM KCl, 10 mM MgCl₂, 5 mM β-glycerophosphate, 100 μM Na Vanadate, 4 μM ATP, 500 nCi [γ-³³P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5× (20 μL of 30 nM and 1.5 μM, respectively) for 15 minutes, and the assay was initiated by the addition of 10 μL of 10 μM ATP. Following the 60-minute incubation, the assay mixtures were quenched by the addition of 100 μL of 25% TCA, the plate was mixed on a rotary shaker for 1 minute, and the product was captured on a Perkin-Elmer GF/B filter plate using a Tomtec Mach III Harvester. After sealing the bottom of the plate, 35 μL of Bio-Safe II (Research Products International) scintillation cocktail were added to each well and the plate was top-sealed and counted in a Topcount NXT (Packard).

The compounds of Examples 1-13 and 15-23 were tested in the above assay and found to have an IC₅₀ of less than 1 μM. The compounds of Examples 1-7, 9-13 and 15-23 were tested in the above assay and found to have an IC₅₀ of less than 500 nM.

### Example A1

### Cellular ERK 1/2 Phosphorylation Assay

Inhibition of basal ERK1/2 phosphorylation was determined by the following in vitro cellular proliferation assay, which comprises incubating cells with a compound of Formula II for 1 hour and quantifying the fluorescent pERK signal on fixed cells and normalizing to total ERK signal.

Materials and Methods: Malme-3M cells were obtained from ATCC and grown in RPMI-1640 supplemented with 10% fetal bovine serum. Cells were plated in 96-well plates at 24,000 cells/well and allowed to attach for 16-20 hours at 37° C., 5% CO₂. The media was removed, and DMSO-diluted compounds were added in RPMI-1640 at a final concentration of 1% DMSO. The cells were incubated with the compounds for 1 hour at 37° C., 5% CO₂. The cells were washed with PBS and fixed in 3.7% formaldehyde in PBS for 15 minutes. This was followed by washing in PBS/0.05% Tween20 and permeabilizing in −20° C. 100% MeOH for 15 minutes. Cells were washed in PBS/0.05% Tween20 then blocked in Odyssey blocking buffer (LI-COR Biosciences) for 1 hour. Antibodies to phosphorylated ERK (1:400, Cell Signaling #9106, monoclonal) and total ERK (1:400, Santa Cruz Biotechnology #sc-94, polyclonal) were added to the cells and incubated 16-20 hours at 4° C. After washing with PBS/0.05% Tween20, the cells were incubated with fluorescently-labeled secondary antibodies (1:1000 goat anti-rabbit IgG-IRDye800, Rockland and 1:500 goat anti-mouse IgG-Alexa Fluor 680, Molecular Probes) for an additional hour. Cells were then washed and analyzed for fluorescence at both wavelengths using the Odyssey Infrared Imaging System (LI-COR Biosciences). Phosphorylated ERK signal was normalized to total ERK signal.

The compounds of Examples 1-5, 7-13, 16, 17 and 19-22 were tested in the above assay and found to have an IC₅₀ of less than 1 μM. The compounds of Examples 1-5, 9-13, 16, 17, 19 and 22 were tested in the above assay and found to have an IC₅₀ of less than 500 nM. By way of example, the following compounds had the following IC₅₀ when measured in the above assay (μM): Example 3 (0.0135), Example 5 (0.0479), Example 19 (0.1765) and Example 21 (0.5221).

### Example B

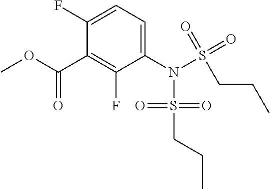

### Methyl 2,6-difluoro-3-(N-(propylsulfonyl)propylsulfonamido)benzoate

Step A: A 1 L flask was charged with 2,6-difluoro-3-nitrobenzoic acid (17.0 g, 83.7 mmol) and MeOH (170 mL, 0.5M). The flask was placed in a cold water bath, and an addition funnel charged with a 2M solution of trimethylsilyl (“TMS”) diazomethane in hexanes (209 mL, 419 mmol) was attached to the flask. The TMS diazomethane solution was added slowly to the reaction flask over the course of 2 hours. A large excess of reagent was required in order for the reaction to reach completion as determined by the ceased evolution of N₂ upon further addition of reagent. The volatiles were removed in vacuo to afford methyl 2,6-difluoro-3-nitrobenzoate as a solid (18.2 g). The material was taken directly onto Step B.

Step B: 10% (wt.) Pd on activated carbon (4.46 g, 4.19 mmol) was added to a 1 L flask charged with methyl 2,6-difluoro-3-nitrobenzoate (18.2 g, 83.8 mmol) under a nitrogen atmosphere. EtOH (350 mL, 0.25 M) was added, and then H₂ was passed through the reaction mixture for 15 minutes. The reaction mixture was stirred under two H₂ balloons overnight. The following day the reaction mixture was re-flushed with fresh H₂ balloons and stirred an additional 4 hours. Upon consumption of the starting material and intermediate hydroxylamine as determined by TLC, N₂ gas was flushed through the reaction mixture. The mixture was then filtered through glass microfibre filter (“GF/F”) paper twice. The volatiles were removed to afford methyl 3-amino-2,6-difluorobenzoate as an oil (15.66 g). The material was taken directly onto the next step.

Step C: Propane-1-sulfonyl chloride (23.46 mL, 209.3 mmol) was slowly added to a solution of methyl 3-amino-2,6-difluorobenzoate (15.66 g, 83.7 mmol) and triethylamine (35.00 mL, 251.1 mmol) in CH₂Cl₂ (175 mL, 0.5 M) maintained in a cool water bath. The reaction mixture was stirred for 1 hour at room temperature. Water (300 mL) was added, and the organic layer was separated, washed with water (2×300 mL), brine (200 mL), then dried (Na₂SO₄), filtered and concentrated to an oil. The crude product was purified by column chromatography, eluting with 15% ethyl acetate (“EtOAc”)/hexane. The isolated fractions were triturated with hexanes to afford methyl 2,6-difluoro-3-(N-(propylsulfonyl)propyl-sulfonamido)benzoate as a solid (24.4 g, 73% yield for 3 steps). ¹H NMR (400 MHz, CDCl₃) δ 7.52-7.45 (m, 1H), 7.08-7.02 (m, 1H), 3.97 (s, 3H), 3.68-3.59 (m, 2H), 3.53-3.45 (m, 2H), 2.02-1.89 (m, 4H), 1.10 (t, J=7.4 Hz, 6H). m/z (APCI-neg) M-(SO₂Pr)=292.2.

### Example C

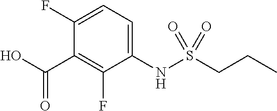

### 2,6-Difluoro-3-(propylsulfonamido)benzoic acid

A 1N aqueous NaOH solution (150 mL, 150 mmol) was added to a solution of methyl 2,6-difluoro-3-(N-(propylsulfonyl)propylsulfonamido)benzoate (20.0 g, 50.1 mmol) in 4:1 THF/MeOH (250 mL, 0.2M). The reaction mixture was stirred at room temperature overnight. The majority of the organic solvents were then removed in vacuo (water bath temperature 35° C.). 1N HCl (150 mL) was slowly added to the mixture, and the resulting solid was filtered and rinsed with water (4×50 mL). The material was then washed with Et₂O (4×15 mL) to give 2,6-difluoro-3-(propylsulfonamido)benzoic acid as a solid (10.7 g, 77% yield). ¹H NMR (400 MHz, DMSO-d₆) δ 9.74 (s, 1H), 7.57-7.50 (m, 1H), 7.23-7.17 (m, 1H), 3.11-3.06 (m, 2H), 1.79-1.69 (m, 2H), 0.98 (t, J=7.4 Hz, 3H). m/z (APCI-neg) M−1=278.0.

### Example D

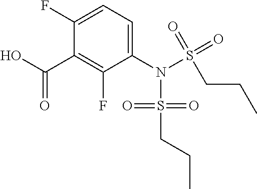

### 2,6-Difluoro-3-(N-(propylsulfonyl)propylsulfonamido)benzoic acid

Propane-1-sulfonyl chloride (1.225 mL, 10.92 mmol) was added to a mixture of 3-amino-2,6-difluorobenzoic acid (0.573 g, 3.310 mmol), triethylamine (2.030 mL, 14.56 mmol) and CH₂Cl₂ (17 mL, 0.2M) cooled to 0° C. The reaction mixture was allowed to warm to room temperature and stirred for 1 hour. The mixture was then partitioned between saturated NaHCO₃ (100 mL) and ethyl acetate (75 mL). The aqueous layer was washed with ethyl acetate (50 mL) and then acidified with concentrated HCl to a pH of about 1. The acidified aqueous layer was extracted with ethyl acetate (2×50 mL), and the combined ethyl acetate extracts were dried (Na₂SO₄), filtered and concentrated. The resulting residue was triturated with hexanes to afford 2,6-difluoro-3-(N-(propylsulfonyl)propyl-sulfonamido)-benzoic acid as a solid (0.948 g, 74% yield). ¹H NMR (400 MHz, DMSO-d₆) δ 7.90-7.84 (m, 1H), 7.39-7.34 (m, 1H), 3.73-3.58 (m, 4H), 1.88-1.74 (m, 4H), 1.01 (t, J=7.5 Hz, 6H). m/z (APCI-neg) M-(SO2Pr)=278.1.

### Example E

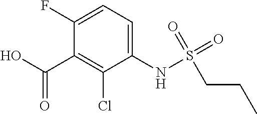

### 2-Chloro-6-fluoro-3-(propylsulfonamido)benzoic acid

Step A: Into a 20-L 4-neck round flask was placed a solution of 2-chloro-4-fluorobenzenamine (1300 g, 8.82 mol, 1.00 equiv, 99%) in toluene (10 L), 4-methylbenzenesulfonic acid (3.1 g, 17.84 mmol, 99%), and hexane-2,5-dione (1222.5 g, 10.62 mol, 1.20 equiv, 99%). The resulting solution was heated to reflux for 1 h in an oil bath and cooled. The pH value of the solution was adjusted to 8 with sodium carbonate (1 mol/L). The resulting mixture was washed with 1×5000 mL of water and concentrated under vacuum. The crude product was purified by distillation and the fraction was collected at 140° C. to afford 1-(2-chloro-4-fluorophenyl)-2,5-dimethyl-1H-pyrrole (1700 g, yield: 85%).

Step B: Into a 5000-mL 4-necked round-bottom flask purged and maintained with an inert atmosphere of nitrogen was placed a solution of 1-(2-chloro-4-fluorophenyl)-2,5-dimethyl-1H-pyrrole (390 g, 1.65 mol, 1.00 equiv, 95%) in tetrahydrofuran (2000 mL). The reaction vessel was cooled to −78° C. To the above reaction vessel was added n-BuLi (800 mL, 1.10 equiv, 2.5%) dropwise with stirring over 80 minutes and methyl carbonochloridate (215.5 g, 2.27 mol, 1.20 equiv, 99%) dropwise with stirring over 90 minutes. The reaction solution was further stirred for 60 minutes at −78° C. and quenched by the addition of 1000 mL of NH₄Cl/water. The resulting solution was extracted with 1500 mL of ethyl acetate. The organic layers were combined, washed with 1×1500 mL of water and 1×1500 mL of sodium chloride(aq), dried over anhydrous magnesium sulfate, and concentrated under vacuum to afford methyl 2-chloro-3-(2,5-dimethyl-1H-pyrrol-1-yl)-6-fluorobenzoate (crude, 566.7 g).

Step C: Into five 5000-mL 4-neck round-bottom flasks was placed a solution of methyl 2-chloro-3-(2,5-dimethyl-1H-pyrrol-1-yl)-6-fluorobenzoate (1500 g, 5.05 mol, 1.00 equiv, 95%) in ethanol/H₂O (7500/2500 mL), NH₂OH—HCl (5520 g, 79.20 mol, 15.00 equiv, 99%), and triethylamine (2140 g, 20.98 mol, 4.00 equiv, 99%). The resulting solution was refluxed for 18 h in an oil bath, cooled to room temperature, concentrated, and extracted with 3×3000 mL of ethyl acetate. The organic layers were combined, dried over anhydrous sodium sulfate, and concentrated under vacuum. The residue was purified using a silica gel column eluting with PE:EA (20:1-10:1) to afford methyl 3-amino-2-chloro-6-fluorobenzoate (980 g, yield: 95%).

Step D: Into four 5000-mL 4-neck round-bottom flasks was placed a solution of methyl 3-amino-2-chloro-6-fluorobenzoate (980 g, 4.76 mol, 1.00 equiv, 99%) in dichloromethane (8000 mL). Triethylamine (1454 g, 14.25 mol, 3.00 equiv, 99%) was added dropwise with stirring at 0° C. over 80 minutes followed by the addition of propane-1-sulfonyl chloride (1725 g, 11.94 mol, 2.50 equiv, 99%). The resulting solution was stirred at room temperature for 2 h, diluted with 1000 mL of water. The organic layer was washed with 1×1000 mL of hydrogen chloride and 1×1000 mL of water, dried over sodium sulfate, and concentrated to afford methyl 2-chloro-6-fluoro-3-(propylsulfonamido)benzoate as a brown solid (1500 g, 97%).

Step E: Into a 10000-mL 4-necked round-bottom flask was placed a solution of methyl 2-chloro-6-fluoro-3-(propylsulfonamido)benzoate (1500 g, 4.61 mol, 1.00 equiv, 95%) in tetrahydrofuran/H₂O (3000/3000 mL) and potassium hydroxide (1000 g, 17.68 mol, 4.50 equiv, 99%). The resulting solution was refluxed for 2 hours, cooled to room temperature and extracted with 3×2000 mL of ethyl acetate. The aqueous layers were combined and the pH was adjusted to 2 with hydrogen chloride (2 mol/L). The resulting solution was extracted with 2×3000 mL of dichloromethane. The organic layers were combined, dried over anhydrous sodium sulfate and concentrated to afford 2-chloro-6-fluoro-3-(propylsulfonamido)benzoic acid (517.5 g, yield: 37%). ¹H NMR (400 MHz, CDCl₃): δ 1.058-1.096 (m, J=15.2 Hz, 3H), 1.856-1.933 (m, 2H), 3.073-3.112 (m, 2H); 6.811 (1H, s), 7.156-7.199 (d, J=17.2 Hz, 1H), 7.827-7.863 (d, J=14.4 Hz, 1H); (ES, m/z): [M+H]⁺ 296.

### Example F

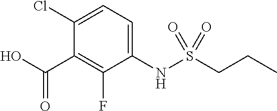

### 6-Chloro-2-fluoro-3-(propylsulfonamido) benzoic acid

Step A: A flame dried flask equipped with a stir bar and rubber septum was charged with 4-chloro-2-fluoroaniline (5.00 g, 34.35 mmol) and anhydrous THF (170 mL). This solution was chilled to −78° C., and n-BuLi (14.7 mL, 1.07 eq. of 2.5M solution in hexanes) was then added over a 15 minute period. This mixture was stirred at −78° C. for 20 minutes, and then a THF solution (25 mL) of 1,2-bis(chlorodimethylsilyl)ethane (7.76 g, 1.05 eq.) was added slowly (over a 10 minute period) to the reaction mixture. This was stirred for 1 hour, and then 2.5M n-BuLi in hexanes (15.11 mL, 1.1 eq.) was added slowly. After allowing the mixture to warm to room temperature for one hour, the mixture was chilled back to −78° C. A third allotment of n-BuLi (15.66 mL, 1.14 eq.) was added slowly, and the mixture was stirred at −78° C. for 75 minutes. Benzyl chloroformate (7.40 g, 1.2 eq.) was then added slowly, and the mixture was stirred at −78° C. for one hour. The cooling bath was then removed. The mixture was allowed to warm for 30 minutes and then quenched with water (70 mL) and concentrated HCl (25 mL). The mixture was allowed to continue to warm to room temperature. The mixture was then extracted with EtOAc. The extracts were washed twice with a saturated NaHCO₃ solution, once with water, dried over sodium sulfate and concentrated. The resulting residue was purified via silica gel column chromatography (30% ethyl acetate/hexane) to produce benzyl 3-amino-6-chloro-2-fluorobenzoate (4.3 g, 45%) as an oil. ¹H NMR (DMSO-d₆, 400 MHz) δ 7.37-7.48 (m, 5H), 7.07 (dd, J=8 Hz, 2 Hz, 1H), 6.87 (t, J=8 Hz, 1H), 5.61 (br s, 2H), 5.40 (s, 2H).

Step B: Benzyl 3-amino-6-chloro-2-fluorobenzoate (4.3 g, 15.37 mmol) was dissolved in dry dichloromethane (270 mL). Triethylamine (5.36 mL, 2.5 eq.) was added, and the mixture was chilled to 0° C. Propane-1-sulfonyl chloride (3.63 mL, 32.3 mmol, 2.1 eq.) was then added via syringe, and a precipitate resulted. Once the addition was complete, the mixture was allowed to warm to room temperature, and the starting material was consumed as determined by TLC (3:1 hexane:ethyl acetate). The mixture was then diluted with dichloromethane (200 mL), washed with 2M aqueous HCl (2×100 mL), saturated NaHCO₃ solution, dried over sodium sulfate and concentrated. The resulting residue was purified via silica gel column chromatography (40% ethyl acetate/hexane) to produce benzyl 6-chloro-2-fluoro-3-(N-(propylsulfonyl)propylsulfonamido)benzoate (5.5 g, 72%) as an oil that slowly solidified upon standing. ¹H NMR (CDCl₃, 400 MHz) δ 7.28-7.45 (m, 7H), 5.42 (s, 2H), 3.58-3.66 (m, 2H), 3.43-3.52 (m, 2H), 1.08 (t, J=8 Hz, 6H).

Step C: Benzyl 6-chloro-2-fluoro-3-(N-(propylsulfonyl)propylsulfonamido) benzoate (5.4 g, 10.98 mmol) was dissolved in THF (100 mL) and 1M aqueous KOH (100 mL). This mixture was refluxed for 16 hours and then allowed to cool to room temperature. The mixture was then acidified to a pH of 2 with 2M aqueous HCl and extracted with EtOAc (2×). The extracts were washed with water, dried over sodium sulfate and concentrated to a solid that was triturated with hexanes/ether to give 6-chloro-2-fluoro-3-(propylsulfonamido)benzoic acid (2.2 g, 68%) as a solid. ¹H NMR (DMSO-d₆, 400 MHz) δ 9.93 (s, 1H), 7.49 (t, J=8 Hz, 1H), 7.38 (dd, J=8 Hz, 2 Hz, 1H), 3.11-3.16 (m, 2H), 1.68-1.78 (m, 2H), 0.97 (t, J=8 Hz, 3H).

### Example G

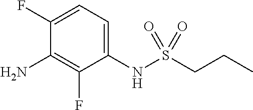

### N-(3-Amino-2,4-difluorophenyl)propane-1-sulfonamide

To a solution of 2,6-difluoro-3-(propylsulfonamido)benzoic acid (4.078 g, 14.6 mmol) in THF (60 mL) was added triethylamine (4.68 mL, 33.59 mmol) and diphenylphosphonic azide (3.73 mL, 16.79 mmol). The reaction mixture was stirred at room temperature for 3 hours and then warmed to 80° C. for 2 hours. Water (10 mL) was added, and the mixture stirred at 80° C. for 15 hours. The reaction mixture was diluted with 300 mL of EtOAc, and the organic layer was washed with saturated aq. NaHCO₃ solution and brine. The solvent was removed under reduced pressure, and the residual purified via silica gel column chromatography eluting with 30/70 EtOAc/hexane to obtain 2.03 g (55%) of the title compound. ¹H NMR (400 MHz, DMSO-d₆) δ 9.32 (s, 1H), 6.90-6.80 (m, 1H), 6.51 (td, J=8.7 Hz, 5.5 Hz, 1H), 5.28 (s, 2H), 3.05-2.96 (m, 2H), 1.82-1.64 (m, 2H), 1.01-0.90 (m, 3H). LC/MS: m/z 251.1 [M+1].

### Example H

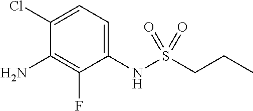

### N-(3-Amino-4-chloro-2-fluorophenyl)propane-1-sulfonamide

The compound was made using the procedure described in Example G using 6-chloro-2-fluoro-3-(propylsulfonamido)benzoic acid instead of 2,6-difluoro-3-(propylsulfonamido)benzoic acid as starting material. ¹H NMR (500 MHz, DMSO-d₆) δ 9.54 (s, 1H), 7.02 (d, 1H), 6.58 (t, 1H), 5.50 (s, 2H), 3.09-2.95 (t, 2H), 1.81-1.64 (sx, 2H), 0.96 (t, 3H). LC/MS: m/z 267.1 [M+1].

### Example I

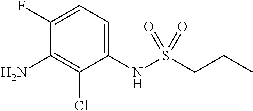

### N-(3-Amino-2-chloro-4-fluorophenyl)propane-1-sulfonamide

The compound was made using the procedure described in Example G using 2-chloro-6-fluoro-3-(propylsulfonamido)benzoic acid instead of 2,6-difluoro-3-(propylsulfonamido)benzoic acid as starting material. ¹H NMR (400 MHz, DMSO-d₆) δ 9.20 (s, 1H), 7.28-6.99 (m, 1H), 6.63 (td, J=8.7 Hz, 5.5 Hz, 1H), 5.45 (s, 2H), 3.07-2.99 (m, 2H), 1.88-1.69 (m, 2H), 1.03-0.95 (m, 3H). LC/MS: m/z 267.1 [M+1].

### Example J

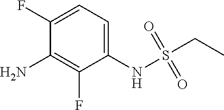

### N-(3-Amino-2,4-difluorophenyl)ethanesulfonamide

The compound was made using the procedure described in Example G using 2,6-difluoro-3-(ethylsulfonamido)benzoic acid instead of 2,6-difluoro-3-(propylsulfonamido)benzoic acid as starting material. ¹H NMR (400 MHz, DMSO-d₆) δ 9.37 (d, J=9.6 Hz, 1H), 6.86 (ddd, J=10.7 Hz, 9.1 Hz, 1.9 Hz, 1H), 6.52 (td, J=8.7 Hz, 5.5 Hz, 1H), 5.28 (s, 2H), 3.03 (q, J=7.3 Hz, 2H), 1.25 (td, J=7.3 Hz, 2.5 Hz, 3H). LC/MS: m/z 237.1 [M+1].

### Example K

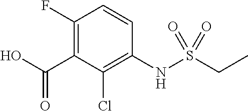

### 2-Chloro-6-fluoro-3-(ethylsulfonamido)benzoic acid

The compound was made using the procedures described in Example E using ethanesulfonyl chloride instead of propane-1-sulfonyl chloride. ¹H NMR (500 MHz, DMSO) 14.16 (br s, 1H), 9.89 (s, 1H), 7.49 (t, J=8.6 Hz, 1H), 7.38 (dd, J=8.8, 1.0 Hz, 1H), 3.16 (q, J=7.3 Hz, 3H), 1.25 (t, J=7.3 Hz, 3H).

### Example L

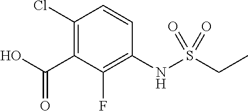

### 6-Chloro-2-fluoro-3-(ethylsulfonamido)benzoic acid

The compound was made using the procedures described in Example F using ethanesulfonyl chloride instead of propane-1-sulfonyl chloride. ¹H NMR (400 MHz, DMSO) δ 9.60 (s, 1H), 7.54 (dd, J=9.0, 5.7 Hz, 1H), 7.35 (t, J=8.8 Hz, 1H), 3.14 (q, J=7.3 Hz, 2H), 1.28 (t, J=7.3 Hz, 3H), —COOH proton not visible.

### Example M

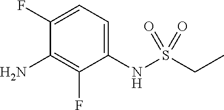

### N-(3-Amino-2,4-difluorophenyl)ethanesulfonamide

The compound was made using the procedure described in Example G using 2,6-difluoro-3-(ethylsulfonamido)benzoic acid instead of 2,6-difluoro-3-(propylsulfonamido)benzoic acid as starting material. ¹H NMR (400 MHz, DMSO-d₆) δ 9.37 (d, J=9.6 Hz, 1H), 6.86 (ddd, J=10.7 Hz, 9.1 Hz, 1.9 Hz, 1H), 6.52 (td, J=8.7 Hz, 5.5 Hz, 1H), 5.28 (s, 2H), 3.03 (q, J=7.3 Hz, 2H), 1.25 (td, J=7.3 Hz, 2.5 Hz, 3H). LC/MS: m/z 237.1 [M+1].

### Example N

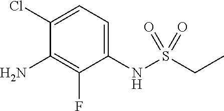

### N-(3-Amino-4-chloro-2-fluorophenyl)ethanesulfonamide

The compound was made using the procedure described in Example G using 6-chloro-3-(ethylsulfonamido)-2-fluorobenzoic acid instead of 2,6-difluoro-3-(propylsulfonamido)benzoic acid as starting material. ¹H NMR (500 MHz, DMSO-d₆) δ 9.48 (s, 1H), 7.02 (dd, J=8.8 Hz, 1.7 Hz, 1H), 6.59 (t, J=8.3 Hz, 1H), 5.44 (s, 2H), 3.07 (q, J=7.3 Hz, 2H), 1.24 (t, J=7.3 Hz, 3H). LC/MS: m/z 253.2 [M+1].

### Example O

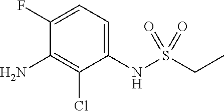

### N-(3-Amino-2-chloro-4-fluorophenyl)ethanesulfonamide

2-Chloro-6-fluoro-3-(ethylsulfonamido)benzoic acid (3.3 g, 12.0 mmol) was treated with thionyl chloride (21.0 mL, 0.29 mmol) and heated at reflux for 15 hours. The reaction mixture was concentrated and then azeotrophed with toluene (2×20 mL). The residue was treated with a solution of sodium azide (3.1 g, 48.0 mmol) dissolved in water (20 mL) and acetone (20 mL). After stirring at room temperature for 1 hour, the intermediate acyl azide was extracted into ethyl acetate (2×25 mL), dried with magnesium sulfate and concentrated. The residue was dissolved in dioxane (40 mL) and water (5 mL) and heated to reflux for 3 hours. After cooling to room temperature, the product was extracted into dichloromethane (2×25 mL), dried with magnesium sulfate and concentrated. The residue was purified by flash silica gel chromatography (2-30% isopropanol in dichloromethane) to afford N-(3-amino-2-chloro-4-fluorophenyl)ethanesulfonamide. (2.0 g, 66%). ¹H NMR (400 MHz, DMSO-d₆) δ 9.15 (s, 1H), 7.02 (dd, J=10.7 Hz, 8.8 Hz, 1H), 6.64 (dd, J=8.8 Hz, 5.1 Hz, 1H), 5.45 (s, 2H), 3.06 (q, J=7.3 Hz, 2H), 0.96 (t, J=7.3 Hz, 3H). LC/MS: m/z 253.0 [M+1].

### Example P

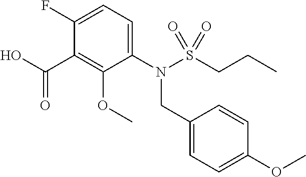

### 6-Fluoro-2-methoxy-3-(N-(4-methoxybenzyl)propylsulfonamido)benzoic acid

Step A: A 250 mL round bottom flask was charged with methyl 2,6-difluoro-3-nitrobenzoate (10.03 g, 46.18 mmol) and methanol (60 mL, 1000 mmol) and was then cooled over a brine/ice bath at −4° C. for 20 minutes. A 5 M solution of sodium methoxide in methanol (11.98 mL, 59.88 mmol) was added to this solution drop wise over 20 minutes while maintaining the reaction temperature at −4° C. over the course of the addition. The reaction mixture was allowed to stir overnight, gradually rising to room temperature. The methanol was removed under reduced pressure and the residual oil quenched with a saturated aqueous solution of potassium bicarbonate (250 mL). The organic layer was saved and the aqueous layer extracted twice with ethyl acetate (250 mL). The combined organic layers were washed once with brine, dried over magnesium sulfate, filtered, and concentrated. The crude product was purified via flash chromatography (330 g ISCO column) using a gradient of 0-50% ethyl acetate: heptane to yield methyl 6-fluoro-2-methoxy-3-nitrobenzoate as an oil (3.37 g. 32%). ¹H NMR (400 MHz, DMSO-d₆) δ=8.23 (dd, J=9.3, 5.9, 1H), 7.39 (t, J=8.9, 1H), 3.94 (s, 3H), 3.89 (s, 3H).

Step B: A 250 mL round bottom flask was charged with methyl 6-fluoro-2-methoxy-3-nitrobenzoate (3.37 g, 14.71 mmol) dissolved in methanol (125 mL, 3080 mmol). Nitrogen was passed through the reaction mixture, and 10% palladium on activated carbon (1.3 g, 1.2 mmol) was added. The flask was capped and evacuated and then allowed to stir for 60 hours under an atmosphere of hydrogen at ambient temperature and pressure. The mixture was then filtered through Celite® to remove the solid catalyst and washed with methanol (500 mL). The filtrate was concentrated to give methyl 3-amino-6-fluoro-2-methoxybenzoate as an oil (2.95 g, 100%). ¹H NMR (400 MHz, DMSO-d₆) δ=6.74-6.84 (m, 2H), 4.98 (s, 2H), 3.85 (s, 3H), 3.68 (s, 3H).

Step C: A 250 mL round bottom flask was charged with a solution of methyl 3-amino-6-fluoro-2-methoxybenzoate (3.656 g, 18.36 mmol) in methylene chloride (100 mL). To this reaction mixture was added a solution of 4-dimethylaminopyridine (113 mg, 0.925 mmol), pyridine (7.45 mL, 92.1 mmol) and propane-1-sulfonyl chloride (8.25 mL, 73.6 mmol) in methylene chloride (10 mL) over a course of five minutes. The reaction mixture was stirred at room temperature for 14 hours. After removing the organic solvent under reduced pressure, 100 mL saturated aqueous sodium bicarbonate was added followed by stirring for 10 minutes. The aqueous mixture was extracted with 200 mL ethyl acetate (2×). The combined organic layers were washed with brine, dried over magnesium sulfate, filtered and concentrated. The crude product was purified using flash chromatography, eluting with 0-30% ethyl acetate/heptanes to give methyl 6-fluoro-2-methoxy-3-(propylsulfonamido)benzoate as an oil (4.914 g, 85%). ¹H NMR (400 MHz, DMSO-d₆) δ=9.27 (s, 1H), 7.48 (dd, J=9.1, 6.1, 1H), 7.09 (t, J=9.0, 1H), 3.89 (s, 3.85-3.74 (m, 3H), 3.29 (s, 15H).

Step D: A 100 mL round bottom flask was charged with methyl 6-fluoro-2-methoxy-3-(propylsulfonamido)benzoate (4.91 g, 16.1 mmol) dissolved in N,N-dimethylformamide (16 mL, 210 mmol) and was cooled over an ice/brine bath. Sodium hydride (0.676 g, 16.9 mmol) was added in four portions. After the vigorous bubbling subsided, the reaction mixture was stirred for 1 h at room temperature. The reaction mixture was cooled over an ice/brine bath, and p-methoxybenzyl chloride (2.646 g, 16.90 mmol) was added. The reaction was allowed to warm to room temperature over the next three hours and then as quenched by adding a semi-saturated aqueous ammonium chloride solution (200 mL) at 0° C. After stirring at room temperature overnight, the aqueous layer was discarded and the remaining oil washed with heptanes to remove the mineral oil. The residual oil was dissolved in ethyl acetate, dried over magnesium sulfate, filtered and concentrated to remove the ethyl acetate. The crude product was purified by flash chromatography (120 g column), using a gradient of 0-100% ethyl acetate: heptanes to give methyl 6-fluoro-2-methoxy-3-(N-(4-methoxybenzyl)propylsulfonamido) benzoate as an oil (3.71 g, 57%). ¹H NMR (400 MHz, DMSO-d₆) δ=7.28 (dd, J=9.0, 6.3, 1H), 7.12 (d, J=8.7, 2H), 6.98 (t, J=8.9, 1H), 6.84 (dd, J=6.8, 4.8, 2H), 4.65 (s, 2H), 3.90 (d, J=7.6, 3H), 3.73 (s, 3H), 3.70 (s, 3H), 3.28-3.21 (m, 2H), 1.84-1.70 (m, 2H), 1.00 (q, J=7.2, 3H).

Step E: A 250 mL round bottom flask was charged with methyl 6-fluoro-2-methoxy-3-(N-(4-methoxybenzyl)propylsulfonamido)benzoate (4.42 g, 10.4 mmol) dissolved in tetrahydrofuran (70 mL, 900 mmol). 1M of sodium hydroxide in water (67.8 mL, 67.8 mmol) was added, and the mixture was stirred at 60° C. for 48 hours. After cooling, the THF was removed under reduced pressure. The basic aqueous solution was diluted with water to a volume of 100 mL and then extracted once with ethyl acetate (200 mL). The aqueous layer was acidified with concentrated hydrochloric acid (5 mL) to a pH of 2 and extracted three times with of ethyl acetate (100 mL). The combined organic layers were dried over magnesium sulfate, filtered and concentrated to afford 6-fluoro-2-methoxy-3-(N-(4-methoxybenzyl)propyl-sulfonamido)benzoic acid as a solid (4.2529 g, 99%). ¹H NMR (400 MHz, DMSO-d₆) δ=13.86 (s, 1H), 7.19 (dd, J=8.9, 6.3, 1H), 7.12 (d, J=8.6, 2H), 6.93 (t, J=8.8, 1H), 6.83 (d, J=8.7, 2H), 4.65 (s, 2H), 3.80 (s, 3H), 3.70 (s, 3H), 3.27-3.19 (m, 2H), 1.78 (dd, J=15.3, 7.5, 2H), 1.01 (t, J=7.4, 3H).

### Example Q

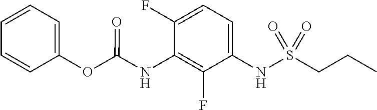

### Phenyl 2,6-difluoro-3-(propylsulfonamido)phenylcarbamate

2,6-Difluoro-3-(propylsulfonamido)benzoic acid (4 g, 14 mmol) was dissolved in 1,4-dioxane (100 mL), and triethylamine (2.2 mL, 16 mmol) and diphenylphosphonic azide (3.4 mL, 16 mmol) were added. The reaction mixture was stirred at room temperature for 3 hours and then added drop-wise to a solution of phenol (15 g, 160 mmol) in 1,4-dioxane (100 mL) at 100° C. The mixture was stirred at 100° C. for 3 hours and then cooled to room temperature. Silica was added, and the mixture was concentrated. Purification via flash chromatography (gradient elution, solvent: 0-30% ethyl acetate in heptanes) yielded phenyl 2,6-difluoro-3-(propylsulfonamido)phenylcarbamate (2.9 g, 55% yield). ¹H NMR (400 MHz, DMSO-d₆) δ 9.85 (s, 1H), 9.68 (s, 1H), 7.49-7.30 (m, 3H), 7.30-7.11 (m, 4H), 3.11-3.03 (m, 2H), 1.83-1.61 (m, 2H), 0.96 (t, J=7.4, 3H).

### Example R

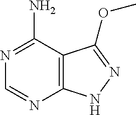

### 3-Methoxy-1H-pyrazolo[3,4-d]pyrimidin-4-amine

Step A: To a mixture of tetracyanoethylene (300 g, 2.34 mol) and urea (47.7 g, 0.79 mol) at rt was added methanol (1 L). The reaction mixture was heated at 35° C. for 20 min, cooled to room temperature, and diluted with diethyl ether (4 L). The mixture was cooled at −78° C. for 3 hours, filtered, washed with cold ether (400 mL) and dried in vacuo to give 2-(dimethoxymethylene)malononitrile (200 g, 62%) as a white solid, which was used directly in the next step.

Step B: To a mixture of 2-(dimethoxymethylene)malononitrile (200 g, 1.45 mol) in H₂O (2.7 L) at rt was added hydrazine monohydrate (78 mL, 1.63 mol). The reaction mixture was stirred for 14 hours, filtered, washed with H₂O (400 mL) and dried in vacuo to afford 5-amino-3-methoxy-1H-pyrazole-4-carbonitrile (140 g, 70%) as a light yellow solid: ¹H NMR (400 MHz, DMSO-d₆) δ 11.04 (bs, 1H), 6.35 (bs, 2H), 3.76 (s, 3H).

Step C: A mixture of 5-amino-3-methoxy-1H-pyrazole-4-carbonitrile (140 g, 1.01 mol) and formamidine acetate (140 g, 1.34 mol) was heated at 145° C. for 1 hour, cooled to rt and diluted with H₂O (1.2 L). The mixture was vigorously stirred for 2 hours and filtered. The solid was washed with methanol (400 mL) and dried in vacuo to afford 3-methoxy-1H-pyrazolo[3,4-d]pyrimidin-4-amine (105 g, 64%) as a brown solid: ¹H NMR (400 MHz, DMSO-d₆) δ 12.31 (bs, 1H), 8.07 (s, 1H), 7.48 (bs, 1H), 6.63 (bs, 1H), 3.94 (s, 3H).

### Example S

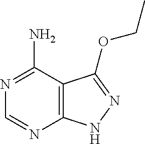

### 3-Ethoxy-1H-pyrazolo[3,4-d]pyrimidin-4-amine

5-Amino-3-ethoxy-1H-pyrazole-4-carbonitrile (1.0 g, 6.57 mmol) and formamidine acetate (3.08 g, 29.6 mmol) were mixed in a microwave vial and heated over a heatgun. The mixture fused, and heating was continued until gas evolution ceased. The mixture was cooled and dissolved in hot aqueous methanol. Decolorizing charcoal was added, and the mixture was filtered hot. The solution was cooled in an ice bath to yield 3-ethoxy-1H-pyrazolo[3,4-d]pyrimidin-4-amine (255 mg, 22% yield) upon filtration. The filtrate was concentrated, and the resulting solids were suspended in water to obtain an additional crop of product (214 mg, 18% yield). ¹H NMR (400 MHz, DMSO-d₆) δ 12.16 (s, 1H), 8.07 (s, 1H), 7.39 (s, 1H), 6.46 (s, 1H), 4.32 (q, J=7.0, 2H), 1.39 (t, J=7.0, 3H).

### Example T

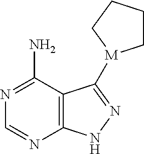

### 3-(Pyrrolidin-1-yl)-1H-pyrazolo[3,4-d]pyrimidin-4-amine

Commercially available 5-amino-3-(pyrrolidin-1-yl)-1H-pyrazole-4-carbonitrile and formamidine acetate were reacted as described in Example O, step C, to yield 3-(pyrrolidin-1-yl)-1H-pyrazolo[3,4-d]pyrimidin-4-amine. ¹H NMR (400 MHz, DMSO-d₆) δ 12.28 (s, 1H), 8.06-8.02 (m, 1H), 6.78 (br s, 2H), 3.38-3.31 (m, 4H), 2.00-1.79 (m, 4H).

### Example U

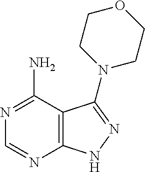

### 3-Morpholino-1H-pyrazolo[3,4-d]pyrimidin-4-amine

Commercially available 5-amino-3-morpholino-1H-pyrazole-4-carbonitrile and formamidine acetate were reacted as described in Example O, step C, to yield 3-morpholino-1H-pyrazolo[3,4-d]pyrimidin-4-amine. ¹H NMR (400 MHz, DMSO-d₆) δ 12.58 (s, 1H), 8.14-8.08 (m, 1H), 6.80 (br s, 2H), 3.80-3.66 (m, 4H), 3.25-3.06 (m, 4H).

### Example 1

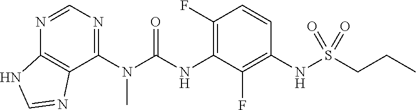

### Propane-1-sulfonic acid {2,4-difluoro-3-[3-methyl-3-(9H-purin-6-yl)-ureido]-phenyl}-amide

2,6-Difluoro-3-(propylsulfonamido)benzoic acid (248 mg, 0.89 mmol) was dissolved in THF (10 mL), and triethylamine (0.248 mL, 2.04 mmol) and diphenylphosphonic azide (0.22 mL, 1.02 mmol) were added. The reaction mixture was stirred at room temperature for 3 hours and then added drop-wise to a solution of N-methyl-9H-purin-6-amine (165.6 mg, 1.11 mmol) in THF (2 mL) at 100° C. The mixture was stirred at 100° C. for 3 hours and then cooled to room temperature, diluted with water, and filtered. The solids were washed with water to yield the title compound (10.1 mg, 3% yield). ¹H NMR (400 MHz, DMSO-d₆) δ 12.04 (s, 1H), 10.13-9.28 (m, 1H), 8.63 (s, 1H), 8.49 (s, 1H), 7.33 (dd, J=14.5, 8.7, 1H), 7.19 (d, J=8.7, 2H), 3.89 (s, 3H), 3.06 (dd, J=16.8, 9.1, 2H), 1.92-1.65 (m, 2H), 0.98 (t, J=7.4, 3H). LC/MS: m/z 426.1 [M+1]; RT=3.56 min.

### Example 2

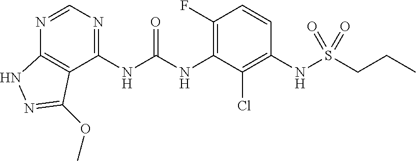

### Propane-1-sulfonic acid {2-chloro-4-fluoro-3-[3-(3-methoxy-1H-pyrazolo[3,4-d]pyrimidin-4-yl)-ureido]-phenyl}-amide

2-Chloro-6-fluoro-3-(propylsulfonamido)benzoic acid (182.3 mg, 0.62 mmol) was dissolved in THF (5 mL), and triethylamine (0.229 mL, 1.61 mmol) and diphenyl-phosphonic azide (0.178 mL, 0.82 mmol) were added. The reaction mixture was stirred at room temperature for 3 hours and then added drop-wise to a solution of 3-methoxy-1H-pyrazolo[3,4-d]pyrimidin-4-amine (34 mg, 0.2 mmol) in THF (2 mL) at 100° C. The reaction mixture was stirred at 100° C. for 3 h, then cooled to room temperature, diluted with water, and filtered. The solids were washed with water to yield the title compound (17.3 mg, 18% yield). ¹H NMR (400 MHz, DMSO-d₆) δ 13.10 (s, 1H), 11.29 (s, 1H), 9.65 (s, 1H), 8.56 (s, 1H), 8.48 (s, 1H), 7.39 (ddd, J=36.4, 13.7, 7.2, 2H), 4.05 (s, 3H), 3.20-2.97 (m, 2H), 1.84-1.65 (m, 2H), 1.02-0.89 (m, 3H). LC/MS: m/z 458.0 [M+1]; RT=3.89 min.

Examples 3-7 listed in Table 1 were prepared applying the procedures described in Example 1 or 2 and using appropriate starting materials.

TABLE 1

| Example | | | MS | ¹H NMR δ |
|---|---|---|---|---|
| no. | Structure | Name | m/z | (400 MHz, DMSO-d6) |
| 3 | 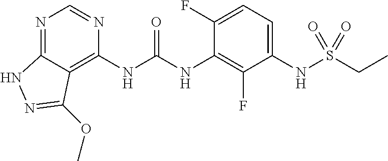 | Ethanesulfonic acid {2,4-difluoro-3-[3-(3- methoxy-1H- pyrazolo[3,4- d]pyrimidin-4-yl)- ureido]-phenyl}-amide | 428.0 | 13.09 (s, 1H), 11.08 (s, 1H), 8.55 (s, 1H), 8.28 (s, 1H), 7.34 (dd, J = 14.6, 8.8, 1H), 7.23-7.08 (m, 2H), 4.00 (s, 3H), 3.05 (q, J = 7.3, 2H), 1.35-1.09 (m, 3H). LC-MS [M + 1] m/z 428.0 |
| 4 | 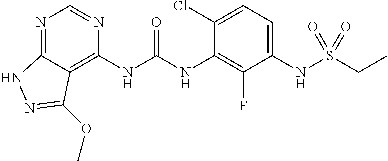 | Ethanesulfonic acid {4- chloro-2-fluoro-3-[3-(3- methoxy-1H- pyrazolo[3,4- d]pyrimidin-4-yl)- ureido]-phenyl}-amide | 444.0 | 13.10 (s, 1H), 11.28 (s, 1H), 9.86 (s, 1H), 8.55 (s, 1H), 8.51 (s, 1H), 7.40 (d, J = 6.7, 2H), 4.13-3.97 (m, 3H), 3.11 (dt, J = 14.5, 7.2, 2H), 1.24 (dt, J = 13.1, 5.9, 3H). LC-MS [M + 1] m/z 444.0 |
| 5 | 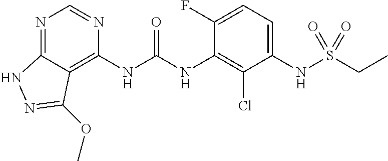 | Ethanesulfonic acid {2- chloro-4-fluoro-3-[3-(3- methoxy-1H- pyrazolo[3,4- d]pyrimidin-4-yl)- ureido]-phenyl}-amide | 444.0 | 13.07 (s, 1H), 11.32 (s, 1H), 9.60 (s, 1H), 8.56 (s, 1H), 8.52 (s, 1H), 7.45 (dd, J = 9.1, 5.3, 1H), 7.40-7.31 (m, 1H), 4.00 (s, 3H), 3.23-3.02 (m, 3H), 1.42-1.14 (m, 3H). LC-MS [M + 1] m/z 444.0 |
| 6 | 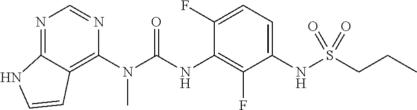 | Propane-1-sulfonic acid {2,4-difluoro-3-[3- methyl-3-(7H- pyrrolo[2,3-d]pyrimidin- 4-yl)-ureido]-phenyl}- amide | 425.2 | (CDCl₃ instead DMSO-d6 used): δ 10.49-10.53 (bs, 1H), 8.42 (s, 1H), 7.73-7.75 (m, 1H), 7.51-7.58 (m, 1H), 7.01- 7.07 (m, 1H), 6.48-6.60 (m, 2H), 5.20-5.35 (bs, 1H), 3.21- 3.25 (m, 3H), 3.06-3.11 (m, 2H), 1.84-1.95 (m, 2H), 1.03- 1.08 (m, 3H) |
| 7 | 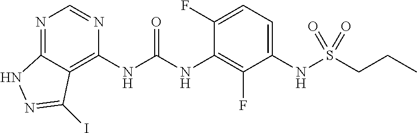 | Propane-1-sulfonic acid {2,4-difluoro-3-[3-(3- iodo-1H-pyrazolo[3,4- d]pyrimidin-4-yl)- ureido]-phenyl}-amide | 538.2 | (CD₃OD instead DMSO-d6 used): δ 8.63 (s, 1H), 7.50 (td, J = 8.9, 5.5 Hz, 1H), 7.18- 7.04 (m, 1H), 3.15-3.00 (m, 2H), 1.94-1.79 (m, 2H), 1.04 (q, J = 7.5 Hz, 3H) |

### Example 8

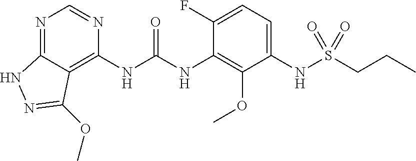

### Propane-1-sulfonic acid {4-fluoro-2-methoxy-3-[3-(3-methoxy-1H-pyrazolo[3,4-d]pyrimidin-4-yl)-ureido]-phenyl}-amide

N-(4-fluoro-2-methoxy-3-(3-(3-methoxy-1H-pyrazolo[3,4-d]pyrimidin-4-yl)ureido)phenyl)-N-(4-methoxybenzyl)propane-1-sulfonamide (89 mg, 0.00016 mol), synthesized using a similar procedure as described in Examples 1 or 2, was dissolved in methylene chloride (5 mL, 0.08 mol), and trifluoroacetic acid (5 mL, 0.06 mol) was added. The reaction mixture was stirred for 30 minutes at room temperature and concentrated under reduced pressure. The resulting white solid was recrystallized from ethyl ether and dried overnight in a vacuum oven to give N-(4-fluoro-2-methoxy-3-(3-(3-methoxy-1H-pyrazolo[3,4-d]pyrimidin-4-yl)ureido)phenyl)propane-1-sulfonamide as an ethyl ether solvate (0.2 eq.) (41 mg, 58%). ¹H NMR (DMSO-d6, 400 MHz) δ 13.09 (s, 1H), 11.09 (s, 1H), 9.20 (s, 1H), 8.56 (s, 1H), 8.40 (s, 1H), 7.30 (dd, J=9.1, 5.7, 1H), 7.07 (t, J=9.2, 1H), 4.05 (s, 3H), 3.82 (s, 3H), 3.14-3.07 (m, 2H), 1.83-1.70 (m, 2H), 0.99 (t, J=7.4, 3H). LC/MS: m/z 454.1 [M+1].

### Example 9

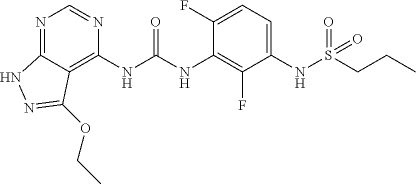

### N-(3-(3-(3-Ethoxy-1H-pyrazolo[3,4-d]pyrimidin-4-yl)ureido)-2,4-difluorophenyl)propane-1-sulfonamide

Phenyl 2,6-difluoro-3-(propylsulfonamido)phenylcarbamate (318 mg, 0.86 mmol) and 3-ethoxy-1H-pyrazolo[3,4-d]pyrimidin-4-amine (310 mg, 1.7 mmol) were suspended in dimethyl sulfoxide (1 mL). The mixture was stirred at 60° C. for 14 hours. The mixture was cooled to room temperature and loaded onto silica gel. Flash chromatography using gradient elution 0-50% (15% methanol in ethyl acetate v/v) in heptanes yielded N-(3-(3-(3-ethoxy-1H-pyrazolo[3,4-d]pyrimidin-4-yl)ureido)-2,4-difluorophenyl)propane-1-sulfonamide as a white powder (115 mg, 29% yield). ¹H NMR (400 MHz, DMSO-d₆) δ 13.06 (s, 1H), 11.15 (s, 1H), 9.69 (s, 1H), 8.55 (s, 1H), 8.41 (s, 1H), 7.37 (dd, J=14.4, 8.8, 1H), 7.21 (t, J=9.2, 1H), 4.43 (q, J=7.0, 2H), 3.14-3.00 (m, 2H), 1.84-1.65 (m, 2H), 1.45 (t, J=7.0, 3H), 0.98 (t, J=7.4, 3H). LC/MS: m/z 456.1 [M+1].

Examples 10-27 listed in Table 2 were prepared applying the procedure described in Example 9 and using appropriate starting materials.

TABLE 2

| Example | | | MS | ¹H NMR δ |
|---|---|---|---|---|
| no. | Structure | Name | m/z | (400 MHz, DMSO-d6) |
| 10 | 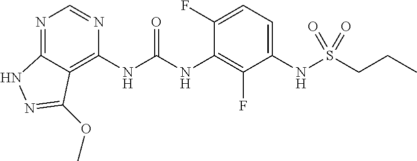 | Propane-1-sulfonic acid {2,4-difluoro-3-[3-(3- methoxy-1H- pyrazolo[3,4- d]pyrimidin-4-yl)- ureido]-phenyl}-amide | 442.1 | 13.09 (s, 1H), 11.10 (s, 1H), 9.81 (s, 1H), 8.52 (d, J = 22.0, 2H), 7.35 (dd, J = 14.1, 8.3, 1H), 7.17 (t, J = 9.1, 1H), 4.05 (s, 3H), 3.05 (s, 2H), 1.86-1.65 (m, 2H), 0.97 (t, J = 7.4, 3H) |
| 11 | 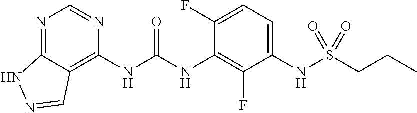 | Propane-1-sulfonic acid {2,4-difluoro-3-[3-(1H- pyrazolo[3,4- d]pyrimidin-4-yl)- ureido]-phenyl}-amide | 412.0 | 13.99 (s, 1H), 11.13 (s, 1H), 11.03 (s, 1H), 9.68 (s, 1H), 8.59 (s, 1H), 8.54 (s, 1H), 7.37 (dd, J = 14.6, 8.8, 1H), 7.20 (t, J = 9.5, 1H), 3.16- 3.01 (m, 2H), 1.84-1.67 (m, 2H), 0.98 (t, J = 7.4, 3H). |
| 12 | 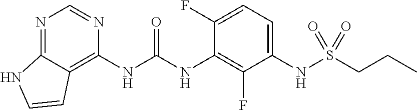 | Propane-1-sulfonic acid {2,4-difluoro-3-[3-(7H- pyrrolo[2,3-d]pyrimidin- 4-yl)-ureido]-phenyl}- amide | 411.0 | 12.11 (s, 1H), 11.63 (s, 1H), 10.49 (s, 1H), 8.44 (s, 1H), 7.40-7.36 (m, 2H), 7.32 (dd, J = 14.6, 8.9, 1H), 7.12 (t, J = 9.2, 1H), 7.03 (d, J = 1.7, 1H), 3.07-2.94 (m, 2H), 1.81-1.66 (m, 2H), 0.97 (t, J = 7.4, 3H) |
| 13 | 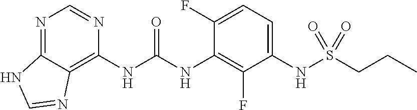 | Propane-1-sulfonic acid {2,4-difluoro-3-[3-(9H- purin-6-yl)-ureido]- phenyl}-amide | 412.0 | 13.98 (s, 1H), 11.15 (s, 1H), 11.01 (s, 1H), 9.65 (s, 1H), 8.57 (s, 1H), 8.52 (s, 1H), 7.35 (dd, J = 14.6, 8.8, 1H), 7.18 (t, J = 9.5, 1H), 3.15- 3.01 (m, 2H), 1.83-1.65 (m, 2H), 0.97 (t, J = 7.4, 3H) |
| 14 | 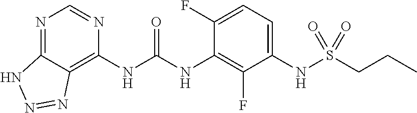 | Propane-1-sulfonic acid {2,4-difluoro-3-[3-(3H- [1,2,3]triazolo[4,5- d]pyrimidin-7-yl)- ureido]-phenyl}-amide | 486.1 | 13.99 (s, 1H), 11.13 (s, 1H), 11.03 (s, 1H), 9.68 (s, 1H), 8.54 (s, 1H), 7.37 (dd, J = 14.6, 8.8, 1H), 7.20 (t, J = 9.5, 1H), 3.16-3.01 (m, 2H), 1.84-1.67 (m, 2H), 0.98 (t, J = 7.4, 3H) |
| 15 | 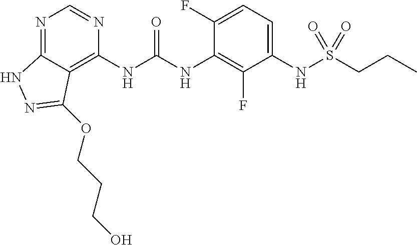 | Propane-1-sulfonic acid {2,4-difluoro-3-{3-[3-(3- hydroxy-propoxy)-1H- pyrazolo[3,4- d]pyrimidin-4-yl]- ureido}-phenyl)-amide | 413.0 | 12.30 (s, 1H), 9.61 (s, 1H), 9.24 (s, 1H), 8.11 (s, 1H), 7.30 (td, J = 8.8, 5.6, 1H), 7.13 (t, J = 9.2, 1H), 4.34 (t, J = 6.1, 2H), 4.29 (t, J = 6.4, 2H), 3.10-2.98 (m, 2H), 2.19-2.05 (m, 2H), 1.83- 1.62 (m, 2H), 0.94 (dt, J = 11.3, 5.8, 3H) |
| 16 | 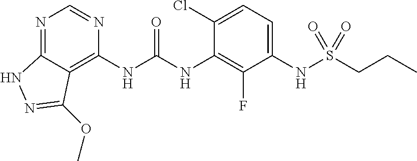 | Propane-1-sulfonic acid {4-chloro-2-fluoro-3-[3- (3-methoxy-1H- pyrazolo[3,4- d]pyrimidin-4-yl)- ureido]-phenyl}-amide | 458.0 | 13.12 (s, 1H), 11.29 (s, 1H), 9.89 (s, 1H), 8.55 (d, J = 8.6, 2H), 7.44-7.36 (m, 2H), 4.05 (s, 3H), 3.18-2.99 (m, 2H), 1.86-1.65 (m, 2H), 0.97 (t, J = 7.4, 3H) |
| 17 | 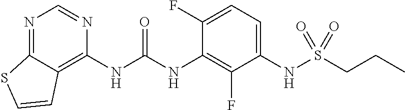 | Propane-1-sulfonic acid [2,4-difluoro-3-(3- thieno[2,3-d]pyrimidin- 4-yl-ureido)-phenyl]- amide | 428.0 | 11.38 (s, 1H), 10.81 (s, 1H), 9.74 (s, 1H), 8.73 (s, 1H), 8.08 (d, J = 6.1, 1H), 7.88 (d, J = 6.0, 1H), 7.36 (td, J = 8.9, 5.7, 1H), 7.19 (t, J = 9.2, 1H), 3.07 (dd, J = 8.7, 6.6, 2H), 1.86-1.65 (m, 2H), 0.95 (dt, J = 13.1, 5.9, 3H) |
| 18 | 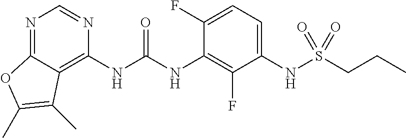 | Propane-1-sulfonic acid {3-[3-(5,6-dimethyl- furo[2,3-d]pyrimidin-4- yl)-ureido]-2,4-difluoro- phenyl}-amide | 440.1 | 10.49 (s, 1H), 9.71 (s, 1H), 9.24 (d, J = 38.3, 1H), 8.56 (s, 1H), 7.34 (td, J = 8.9, 5.8, 1H), 7.17 (t, J = 9.0, 1H), 3.12-2.99 (m, 2H), 2.40 (s, 3H), 2.33 (s, 3H), 1.82-1.68 (m, 2H), 1.03-0.90 (m, 3H) |
| 19 | 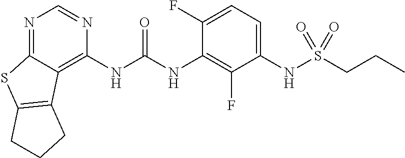 | Propane-1-sulfonic acid {3-[3-(6,7-dihydro-5H- cyclopenta[4,5]thieno[2, 3-d]pyrimidin-4-yl)- ureido]-2,4-difluoro- phenyl}-amide | 468.0 | 10.58 (s, 1H), 9.88 (s, 1H), 8.96 (s, 1H), 8.67 (s, 1H), 8.15 (s, 1H), 7.72-7.54 (m, 2H), 7.46-7.28 (m, 2H), 7.19 (t, J = 8.8, 2H), 3.25- 3.13 (m, 2H), 3.13-2.93 (m, 4H), 1.92-1.60 (m, 4H), 1.09-0.84 (m, 3H) |
| 20 | 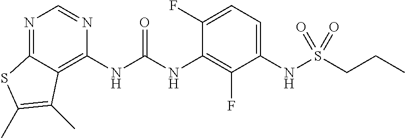 | Propane-1-sulfonic acid {3-[3-(5,6-dimethyl- thieno[2,3-d]pyrimidin- 4-yl)-ureido]-2,4- difluoro-phenyl}-amide | 456.1 | 10.52 (s, 1H), 9.67 (s, 1H), 8.94 (s, 1H), 8.68 (s, 1H), 7.34 (dd, J = 14.6, 8.8, 1H), 7.17 (t, J = 9.4, 1H), 3.15- 2.97 (m, 2H), 2.53 (s, 6H), 1.75 (dq, J = 14.8, 7.4, 2H), 0.98 (t, J = 7.4, 3H) |
| 21 | 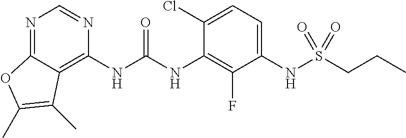 | Propane-1-sulfonic acid {4-chloro-3-[3-(5,6- dimethyl-furo[2,3- d]pyrimidin-4-yl)- ureido]-2-fluoro- phenyl}-amide | 456.0 | 10.72 (s, 1H), 9.85 (s, 1H), 9.16 (s, 1H), 8.57 (s, 1H), 7.49-7.24 (m, 2H), 3.09 (dd, J = 8.7, 6.7, 2H), 2.40 (s, 3H), 2.34 (s, 3H), 1.81-1.66 (m, 2H), 0.97 (t, J = 7.4, 3H) |
| 22 | 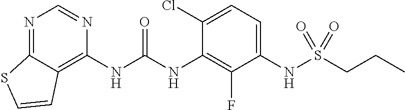 | Propane-1-sulfonic acid [4-chloro-2-fluoro-3-(3- thieno[2,3-d]pyrimidin- 4-yl-ureido)-phenyl]- amide | 444.0 | 11.52 (s, 1H), 10.79 (s, 1H), 8.74 (s, 1H), 8.08 (d, J = 6.1, 1H), 7.88 (d, J = 6.0, 1H), 7.42-7.33 (m, 2H), 3.15- 2.97 (m, 2H), 1.83-1.63 (m, 2H), 0.97 (t, J = 7.4, 3H) |
| 23 | 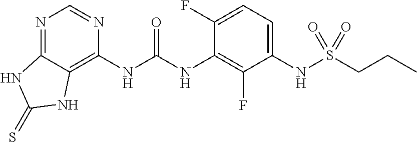 | Propane-1-sulfonic acid {2,4-difluoro-3-[3-(8- thioxo-8,9-dihydro-7H- purin-6-yl)-ureido]- phenyl}-amide | 444.0 | 13.98 (s, 1H), 11.12 (s, 1H), 11.02 (s, 1H), 9.67 (s, 1H), 8.53 (s, 1H), 7.36 (dd, J = 14.6, 8.8, 1H), 7.19 (t, J = 9.5, 1H), 3.16-3.01 (m, 2H), 1.84-1.67 (m, 2H), 0.97 (t, J = 7.4, 3H) |
| 24 | 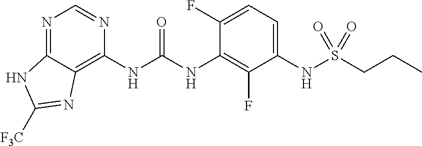 | Propane-1-sulfonic acid {2,4-difluoro-3-[3-(8- trifluoromethyl-9H- purin-6-yl)-ureido]- phenyl}-amide | 480.0 | 12.54 (s, 1H), 9.98 (s, 1H), 9.74 (s, 1H), 8.58 (s, 1H), 8.34 (s, 1H), 7.47-7.37 (m, 1H), 7.28 (dd, J = 9.2, 7.7, 1H), 3.15-3.03 (m, 2H), 1.85-1.72 (m, 2H), 1.00 (t, J = 7.4, 3H) |
| 25 | 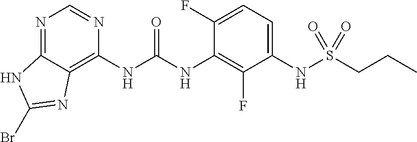 | Propane-1-sulfonic acid {3-[3-(8-bromo-9H- purin-6-yl)-ureido]-2,4- difluoro-phenyl}-amide | 492.0 | 11.86 (s, 1H), 9.68 (s, 1H), 9.44 (s, 1H), 8.28 (s, 1H), 8.14 (s, 1H), 7.37-7.27 (m, 1H), 7.18 (dd, J = 9.2, 7.7, 1H), 3.13-3.01 (m, 2H), 1.81-1.68 (m, 2H), 0.98 (t, J = 7.4, 3H) |
| 26 | 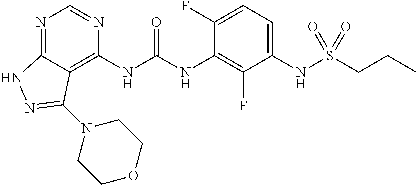 | Propane-1-sulfonic acid {2,4-difluoro-3-[3-(3- morpholin-4-yl-1H- pyrazolo[3,4- d]pyrimidin-4-yl)- ureido]-phenyl}-amide | 497.1 | 12.11 (s, 1H), 9.76 (s, 1H), 8.28 (s, 1H), 7.40 (s, 1H), 7.30 (dd, J = 14.5, 8.8, 1H), 7.14 (t, J = 9.1, 1H), 6.22 (s, 1H), 3.11-2.98 (m, 2H), 2.44-2.28 (m, 4H), 1.85- 1.65 (m, 2H), 0.97 (t, J = 7.4, 3H) |
| 27 | 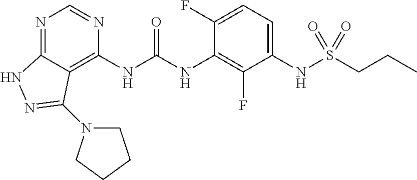 | Propane-1-sulfonic acid {2,4-difluoro-3-[3-(3- pyrrolidin-1-yl-1H- pyrazolo[3,4- d]pyrimidin-4-yl)- ureido]-phenyl}-amide | 481.0 | 11.97 (s, 1H), 8.30 (s, 1H), 7.75 (d, J = 6.6, 1H), 7.27 (dd, J = 14.7, 8.9, 1H), 7.07 (t, J = 8.9, 1H), 6.57 (s, 1H), 6.22 (d, J = 19.6, 1H), 3.92- 3.80 (m, 4H), 3.73 (dt, J = 13.8, 6.9, 2H), 3.06-2.90 (m, 2H), 2.19 (td, J = 14.8, 7.5, 1H), 1.82 (d, J = 4.5, 1H), 1.78-1.64 (m, 2H), 0.96 (t, J = 7.4, 3H) |

### Example α

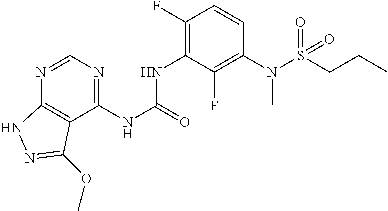

### Propane-1-sulfonic acid {2,4-difluoro-3-[3-(3-methoxy-1H-pyrazolo[3,4-d]pyrimidin-4-yl)-ureido]-phenyl}-methyl-amide

Step A: 2,6-Difluoro-3-(propylsulfonamido)benzoic acid (20.0 g, 71.6 mmol) was dissolved in acetone (400 mL), and potassium carbonate (29.7 g, 215 mmol) was added. After stirring for 5 minutes, methyl iodide (13.4 mL, 215 mmol) was added. The reaction mixture was stirred overnight. The reaction mixture was filtered and the filtrate concentrated. The crude product was partitioned between ethyl acetate and water and the organic layer washed by brine, dried over MgSO₄ and concentrated to dryness. Further removal of solvent under high vacuum afforded methyl 2,6-difluoro-3-(N-methylpropylsulfonamido)benzoate as light yellow solid (17.0 g, 77% yield). ¹H NMR (500 MHz, CDCl₃) δ 7.57 (td, 1H), 7.10-6.89 (m, 1H), 3.96 (s, 3H), 3.31 (s, 3H), 3.13-2.96 (m, 2H), 2.01-1.81 (m, 2H), 1.07 (t, 3H).

Step B: Methyl 2,6-difluoro-3-(N-methylpropylsulfonamido)benzoate (17.0 g, 55 mmol) was dissolved in tetrahydrofuran/methanol/water (1:1:1; 150 mL), and lithium hydroxide (6.96 g, 166 mmol) was added. The mixture was stirred at room temperature for 18 hours and concentrated. The crude product was partitioned between ethyl acetate and water and the aqueous layer acidified with 2M HCl to pH <1, extracted with ethyl acetate, washed by brine, and dried over MgSO₄ Removal of the solvent in vacuo gave 2,6-difluoro-3-(N-methylpropylsulfonamido)benzoic acid as white solid (15.7 g, 97%). ¹H NMR (400 MHz, CDCl₃) δ 7.64 (td, 1H), 7.03 (td, 1H), 3.31 (s, 3H), 3.13-2.97 (m, 2H), 1.99-1.79 (m, 2H), 1.07 (t, 3H).

Step C: 2,6-Difluoro-3-(N-methylpropylsulfonamido)benzoic acid (5.0 g, 17.0 mmol) was dissolved in 1,4-dioxane (40 mL), and triethylamine (2.6 mL, 18.8 mmol), diphenylphosphonic azide (4.0 mL, 18.0 mmol) were added. The mixture was stirred at room temperature for 3 hours and then added dropwise to a solution of water (1.5 mL, 85.2 mmol) in 1,4-dioxane (20 mL) at 95° C. The mixture was stirred at 95° C. for 18 hours and then cooled to room temperature. Water, saturated sodium bicarbonate solution and ethyl acetate were added, and the organic layer was washed with brine, dried over MgSO₄ and concentrated. The crude product was purified using flash chromatography (gradient elution, solvent: 0-40% ethyl acetate in heptanes) to yield N-(3-amino-2,4-difluorophenyl)-N-methylpropane-1-sulfonamide (3.23 g, 72%). ¹H NMR (400 MHz, CDCl₃) δ 6.78 (ddt, 2H), 3.80 (s, 2H), 3.27 (s, 3H), 3.14-2.95 (m, 2H), 2.11-1.78 (m, 2H), 1.06 (t, 3H).

Step D: N-(3-amino-2,4-difluorophenyl)-N-methylpropane-1-sulfonamide (2.0 g, 7.6 mmol) was dissolved in THF (25 ml), and potassium carbonate (4.2 g, 30.3 mmol) and phenyl chloroformate (3.2 mL, 25.0 mmol) were added. The reaction mixture was stirred at room temperature overnight and then concentrated. The residue was partitioned between ethyl acetate/sat. NaHCO₃ solution, and the aqueous layer extracted with ethyl acetate. The combined organic layers were washed with brine, dried over MgSO₄, and concentrated. The crude product was purified using flash chromatography (gradient elution, solvent: 0-40% ethyl acetate in heptanes) to yield phenyl 2,6-difluoro-3-(N-methylpropylsulfonamido)phenylcarbamate (2.8 g, 96%). ¹H NMR (500 MHz, CDCl₃) δ 7.45-7.33 (m, 3H), 7.29-7.13 (m, 3H), 7.06-6.96 (m, 1H), 6.43 (s, 1H), 3.30 (s, 3H), 3.11-3.01 (m, 2H), 1.95-1.82 (m, 2H).

Step E: To a suspension of phenyl 2,6-difluoro-3-(N-methylpropyl-sulfonamido)phenylcarbamate (364 mg, 0.757 mmol) in THF, 3-methoxy-1H-pyrazolo[3,4-d]pyrimidin-4-amine (100 mg, 0.605 mmol), and triethylamine (0.338 mL, 2.42 mmol) were added. The mixture was heated to 50° C. for 3 hours and then cooled. Water (1 mL) was added, followed by extraction with EtOAc. The organic layer was washed with brine, dried over MgSO₄, concentrated, and triturated in ethyl acetate. Filtration and removal of solvent under reduced pressure yielded the title compound as a white solid (115 mg, 41%). ¹H NMR (400 MHz, DMSO-d₆) δ 13.09 (s, 1H), 11.15 (s, 1H), 8.55 (s, 2H), 7.52 (d, 1H), 7.26 (d, 1H), 4.05 (s, 3H), 3.26-3.13 (m, 2H), 1.90-1.53 (m, 2H), 1.00 (t, 3H). LC/MS: m/z 456.1 [M+1]; RT=4.07 min.

### Example β

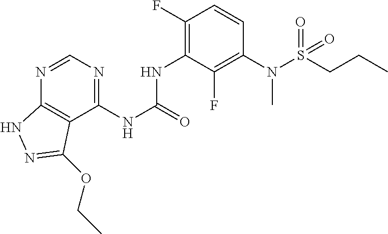

### Propane-1-sulfonic acid {3-[3-(3-ethoxy-1H-pyrazolo[3,4-d]pyrimidin-4-yl)-ureido]-2,4-difluoro-phenyl}-methyl-amide

Using a similar procedure as described for Example Alpha, the title compound was obtained. ¹H NMR (400 MHz, DMSO-d₆) δ 13.07 (s, 1H), 11.19 (s, 1H), 8.54 (s, 1H), 8.43 (s, 1H), 7.52 (d, 1H), 7.26 (t, 1H), 4.43 (q, J=6.9, 2H), 3.25-3.17 (m, 2H), 1.85-1.63 (m, 2H), 1.44 (t, J=7.0, 3H), 1.00 (t, 3H). LC/MS: m/z 470.1 [M+1]; RT=4.36 min.

Table 3 shows the activity of certain compounds of the invention tested in the above B-RAF V600E inhibition assay (Example A).

TABLE 3

| | BRAF V600E |
|---|---|
| Example | IC₅₀ (μM) |
| 1 | 0.0239 |
| 2 | 0.0013 |
| 3 | 0.0034 |
| 4 | 0.0019 |
| 5 | 0.0039 |
| 6 | 0.0570 |
| 7 | 0.0038 |
| 8 | 0.1922 |
| 9 | 0.0009 |
| 10 | 0.0014 |
| 11 | 0.0055 |
| 12 | 0.0562 |
| 13 | 0.0047 |
| 14 | 1.9601 |
| 15 | 0.4280 |
| 16 | 0.0006 |
| 17 | 0.0979 |
| 18 | 0.4307 |
| 19 | 0.0252 |
| 20 | 0.0282 |
| 21 | 0.0908 |
| 22 | 0.0188 |
| 23 | 0.0292 |

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

Specific reference is made to U.S. Provisional Patent Appl. Ser. No. 61/238,108 filed Aug. 28, 2009, which is incorporated by reference herein, in its entirety, for all purposes.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula II: 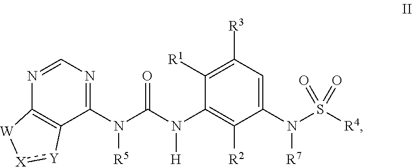
stereoisomers, tautomers, prodrugs and pharmaceutically acceptable salts thereof, wherein:
the dotted line represents an optional double bond;
W is NR⁶, O or S;
X is N or CR⁸;
Y is NR⁹ or CR⁹;
R¹ and R² are independently selected from hydrogen, halogen, CN, C₁-C₃ alkyl and C₁-C₃ alkoxy;
R³ is hydrogen, halogen or C₁-C₃ alkyl;
R⁴ is C₃-C₅ cycloalkyl, C₁-C₆ alkyl, C₂-C₆ alkenyl, C₂-C₆ alkynyl, phenyl, a 5-6 membered heteroaryl or NR¹⁰R¹¹, wherein the cycloalkyl, alkyl, alkenyl, alkynyl, phenyl and heteroaryl are optionally substituted with OR¹², halogen, phenyl, C₃-C₄ cycloalkyl, or C₁-C₄ alkyl optionally substituted with halogen;
R⁵ is hydrogen, C₁-C₃ alkyl, C₂-C₃ alkenyl, C₂-C₃ alkynyl or C₃-C₅ cycloalkyl, wherein R⁵ is optionally substituted with halogen;
R⁶ is hydrogen or C₁-C₃ alkyl;
R⁷ is hydrogen, C₁-C₃ alkyl, C₂-C₃ alkenyl, C₂-C₃ alkynyl or C₃-C₅ cycloalkyl, wherein R⁷ is optionally substituted with halogen;
R⁸ is hydrogen, halogen, oxo, thioxo, C₁-C₆ alkoxy or C₁-C₆ alkyl, wherein each C₁-C₆ alkoxy and C₁-C₃ alkyl is optionally substituted with halogen, OR¹³, SR¹³, NR¹³R¹⁴, C₃-C₆ cycloalkyl, 4-6 membered heterocyclyl, 5-6 membered heteroaryl or phenyl;
R⁹ is hydrogen, halogen, C₁-C₆ alkoxy, C₁-C₆ alkyl, 4-6 membered heterocyclyl, 5-6 membered heteroaryl, phenyl or C₃-C₆ cycloalkyl, wherein said alkoxy, alkyl, heterocyclyl, heteroaryl, phenyl and cycloalkyl are independently optionally substituted with halogen, OR¹⁵, SR¹⁵, NR¹⁵R¹⁶, C₃-C₆ cycloalkyl, 4-6 membered heterocyclyl, 5-6 membered heteroaryl, phenyl or C₁-C₆ alkyl optionally substituted with halogen; or
R⁸ and R⁹ are taken together with the atoms to which they are attached to form a fused 5-6 membered heterocyclyl or C₅-C₆ cycloalkyl;
R¹⁰ and R¹¹ are independently hydrogen or C₁-C₆ alkyl, optionally substituted with halogen; or
R¹⁰ and R¹¹ are independently taken together with the atom to which they are attached to form a 3-6 membered heterocyclyl, optionally substituted with halogen, oxo or C₁-C₃ alkyl;
R¹² is hydrogen or C₁-C₆ alkyl optionally substituted with halogen;
R¹³ and R¹⁴ are independently hydrogen or C₁-C₆ alkyl optionally substituted with halogen; or
R¹³ and R¹⁴ are independently taken together with the atom to which they are attached to form a 3-6 membered heterocyclyl optionally substituted with halogen, oxo or C₁-C₃ alkyl; and
R¹⁵ and R¹⁶ are independently hydrogen or C₁-C₆ alkyl optionally substituted with halogen; or
R¹⁵ and R¹⁶ are independently taken together with the atom to which they are attached to form a 3-6 membered heterocyclyl optionally substituted with halogen, oxo or C₁-C₃ alkyl.

2. A compound of claim 1, wherein W is NR⁶, X is N and Y is CR⁹.

3. A compound of claim 1, wherein W is NR⁶, X is CR⁸ and Y is NR⁹.

4. A compound of claim 1, wherein W is O, X is CR⁸ and Y is CR⁹.

5. A compound of claim 1, wherein W is S, X is CR⁸ and Y is CR⁹.

6. A compound of claim 1, wherein W is NR⁶, and X and Y are N.

7. A compound of any one of claims 1-6, wherein R¹ and R² are independently selected from halogen and C₁-C₃ alkoxy, and R³ is hydrogen.

8. A compound of any one of claims 1-7, wherein R¹ and R² are F and R³ is hydrogen.

9. A compound of any one of claims 1-8, wherein R⁴ is C₁-C₃ alkyl optionally substituted by halogen.

10. A compound of any one of claims 1-9, wherein R⁴ is ethyl or propyl.

11. A compound of any one of claims 1-10, wherein R⁵ is hydrogen.

12. A compound of any one of claims 1-11, wherein R⁷ is hydrogen.

13. A compound of any one of claims 1, 3-5 and 7-12, wherein R⁸ is hydrogen, methyl, —CF₃, thioxo or bromo.

14. A compound of any one of claims 1-5 and 7-13, wherein R⁹ is hydrogen, halogen, C₁-C₆ alkyl, C₁-C₆ alkoxy, 4-6 membered heterocyclyl, 5-6 membered heteroaryl, phenyl or C₃-C₆ cycloalkyl, wherein said alkyl, alkoxy, heterocyclyl, heteroaryl, phenyl and cycloalkyl are independently optionally substituted by halogen, C₁-C₃ alkyl optionally substituted with halogen or OR¹⁵.

15. A compound of any one of claims 1-5 and 7-14, wherein R⁹ is hydrogen, ethoxy, methoxy, 3-hydroxypropoxy, iodo, methyl, morpholinyl or pyrrolidinyl.

16. A compound of any one of claims 1-15, selected from Examples 1-27.

17. A pharmaceutical composition, comprising a compound of any one of claims 1-16, and a pharmaceutically acceptable carrier or excipient.

18. A method of preventing or treating a disease or disorder modulated by b-Raf, comprising administering to a mammal in need of such treatment an effective amount of a compound of any one of claims 1-16.

19. A compound as claimed in any one of claims 1 to 16 for use in therapy.

20. Use of a compound of any one of claims 1 to 16 in the manufacture of a medicament for the treatment of a hyperproliferative disease.
